(19) United States
(12) Patent Application Publication
(10) Pub. No.: US 2024/0002935 A1
(43) Pub. Date: Jan. 4, 2024

(54) GENETIC METHOD FOR DIAGNOSIS AND TREATMENT OF PRE AND POST CORONAVIRUS INFECTIONS

(71) Applicant: Geneticure Inc., Minnetonka, MN (US)

(72) Inventors: Eric Snyder, Rochester, MN (US); Scott C. Snyder, Minnetonka, MN (US); Ryan Sprissler, Tucson, AZ (US)

(21) Appl. No.: 17/995,950

(22) PCT Filed: Apr. 9, 2021
(86) PCT No.: PCT/US2021/026557

§ 371 Date: Oct. 11, 2022

Related U.S. Application Data
(60) Provisional application No. 63/008,676, filed on Apr. 11, 2020.

(51) Int. Cl. C12Q 1/6883; A61K 38/05; A61K 31/138; A61K 31/165; A61K 31/55; A61K 31/401; A61K 31/4178; A61K 31/4184; A61K 45/06; A61K 31/675; A61K 31/417; A61K 31/41

(52) U.S. Cl. CPC C12Q 1/6883; A61K 38/05; A61K 31/138; A61K 31/165; A61K 31/55; A61K 31/401; A61K 31/4178; A61K 31/4184; A61K 45/06; A61K 31/675; A61K 31/417; A61K 31/41; C12Q 2600/156; C12Q 2600/106

(57) ABSTRACT

The present invention is directed to management of coronavirus exposure and/or infection by analyzing a patients 21 SNPs of the patients AGT, renin, AT1R, ACE, ADRB1 and ACE2 genes arnd use of the analysis to determine which pharmaceutical compound(s) associated with treatment of the RAAS system issues should be administered and/or discontinued, if any, to ameliorate the potential for coronavirus infection and/or the severity of coronavirus infection. Typical coronaviruses to be treated include SARS coronavirus, MERS coronavirus and/or Covid-19 coronavirus. Co-administration of anti-viral, quinoline-like and/or quinone-like drugs and ACE2 agonists are also disclosed.

This application claims benefit of priority to the filing date of U.S. Provisional Application Ser. No. 63/008,676 (filed Apr. 11, 2020), the contents of which application is specifically incorporated herein by reference in its entirety.

### BACKGROUND

Coronaviruses are a group of related, common viral agents responsible for disease in mammals and birds. In humans, coronaviruses are responsible at least in part for the common cold as well as other mild illnesses. More serious infections are caused by such corona virus species as MERS coronavirus, SARS coronavirus and covid-19 virus among others. The coronaviruses generally are known to target the certain integral constituents of the membranes of lung epithelial cells. This target provides a means for multiple species of coronavirus to gain cellular entry into human hosts.

In 2019, a novel coronavirus (COVID-19 or C-19; officially called SARS-CoV-2) began to infect humans and resulted in severe respiratory ailment similar to those from severe acute respiratory syndrome coronavirus (SARS-CoV)[1]. Both COVID-19 and SARS-CoV bind to, and utilize, a particular host receptor in order to infect cells which can result in respiratory illness in animals and humans.

Abundant evidence demonstrates that the host receptor for coronaviruses including but not limited to COVID-19, utilizes ACE2 as a pathway of infection in the respiratory tract of humans[1]. The coronavirus is a member of a family of viruses that are single-stranded RNA viruses [2]. The coronavirus utilizes a spike protein that mediates the viral entry into the host cell by binding to a host receptor and fusing the virus and host membranes [3]. Specifically, coronaviruses tend to utilize ACE2 as the host receptor [4].

ACE2 is a relatively newly discovered enzyme in the renin-angiotensin-aldosterone pathway (the RAAS pathway). ACE2 is a homologue of the angiotensin-converting enzyme (ACE) but apparently does not function like ACE.

An objective, therefore, is to ascertain the interrelationship of ACE2 in the RAAS pathway. Another objective is the use of pharmaceutical agents that affect the RAAS pathway to interdict coronavirus infections including but not limited to COVID-19 infection. Yet another objective is determination of genotype influence upon the RAAS pathway and whether genotype affects the ability of coronaviruses such as COVID-19 to infect humans.

### SUMMARY OF THE INVENTION

These and other objects are achieved by the present invention which is directed to development of a set of genotypes that influence coronavirus infections such as but not limited to COVID-19 infection, methods for abating, ameliorating and/or diminishing the ability of coronaviruses such as COVID-19 to infect humans, and methods for treatment of humans infected with coronaviruses such as COVID-19 through use of genotypes and pharmaceutical compounds.

The present invention is directed to embodiments for treatment of persons who have been exposed to coronaviruses such as COVID-19 (C-19) but are not yet infected. These persons may have coronaviruses such as COVID-19 in their respiratory passages including but not limited to sinuses but the coronavirus such as COVID-19 has not yet invaded host cells. These pre-infection persons may be young, old, may have or not have immunodeficient aspects, may have or not have blood pressure issues such as but not limited to hypertension, may have or not have blood sugar issues such as but not limited to type 1 and/or type 2 diabetes, and/or pre-diabetic issues, and may exhibit or not exhibit resistance to coronavirus infection such as for example by the presence of immune system resistance to corona virus and COVID-19 infection such as through antibody or other immune system interaction with coronavirus generally and with COVID-19 specifically.

Described herein are methods and compositions for treatment of pre-infection persons that include determining whether a person has a series of 21 single nucleotide polymorphisms (SNPs) associated with the RAAS pathway. According to embodiments of the invention, certain combinations of these 21 SNPs affect the level of ACE2 in such persons so that the ACE2 level is higher. According to embodiments of the invention, prophylactic treatment of persons with such combinations of SNPs is accomplished by administering one or more RAAS pathway pharmaceuticals that will negatively influence and/or negatively effect and/or cause a decrease in the level of ACE2 in such persons. Such persons may also be administered prophylactically anti-viral pharmaceutical agents that will or may directly or indirectly engage and/or target the coronavirus particles such as COVID-19 particles within the persons so that the particles are at least in part rendered incapable of invading host cells.

According to the invention, embodiments for treatment of post-infection patients comprise determining the same series of 21 SNPs as mentioned above for the pre-infection persons. According to these embodiments of the invention, certain combinations of these 21 SNPs affect the level of ACE2. The various combinations present a mixture of influences on ACE2 levels including a combination of high and low functionality, a combination of high, moderate and no functionality, a combination of high and no functionality or an overall combination of low functionality or a combination that is minimally functional and/or has been usurped by COVID-19 infection in such patients. According to embodiments of the invention, post-infection treatment of persons with such combinations of SNPs is accomplished by administering one or more RAAS pathway pharmaceuticals that will positively influence and/or positively effect and/or cause an increase in the level of ACE2 in such patients. Such patients may also be administered anti-viral pharmaceutical agents that will or may directly or indirectly engage and/or target the COVID-19 particles infecting the patients. Such patients may also be administered antibacterial pharmaceutical agents to combat secondary bacterial infection such as pneumonia.

For pre-infection persons who do not have the certain combinations of SNPs that provide highly functional or moderately functional ACE2 levels, no RAAS pharmaceutical agent is administered. But such persons are to be followed for possible infection. If such persons become infected, the post infection embodiments of the invention are applied.

All post-infection persons exhibiting overt symptoms of COVID-19 infection would be administered one or more RAAS pharmaceutical agents consistent with their underlying RAAS genotype and their cardiovascular phenotype.

The 21 SNPs are polymorphic sequences of the genes coding for angiotensinogen (AGT), Rennin, angiotensin converting enzyme (ACE), angiotensin II receptor type 1 (AT1R), angiotensin converting enzyme 2 (ACE2), and adrenergic receptor beta 1 (ADRB1). These genes are part of the RAAS pathway.

### BRIEF DESCRIPTION OF FIGURES

FIG. 1 provides an algorithm for the pharmacologic considerations for pre-infection ACE2 pathway.

FIG. 2 provides an algorithm for the pharmacologic considerations for post-infection ACE2 pathway.

### DETAILED DESCRIPTION

Scientific evidence indicates that angiotensin converting enzyme 2 (ACE2) is counteractive in nature to the function and activities of ACE. Within the renin-angiotensin aldosterone (RAAS) pathway, renin cleaves angiotensinogen to produce the enzymatic protein angiotensin-I (Ang-I). ACE then converts Ang-I to angiotensin-II (Ang-II) so that ACE is a primary drug target for patients with hypertension. ACE inhibition will lessen Ang-II activity which otherwise produces vasoconstriction and an increase in blood pressure. Ang-II primarily binds to two types of receptors, the angiotensin II type 1 and type 2 receptors (AT1 and AT2 receptors). ACE2 hydrolyzes Ang-I to make angiogenin precursor (1-9) (Ang (1-9)) and hydrolyzes Ang-II to make angiogenin precursor (1-7) (Ang(1-7)). Ang (1-7) is a vasodilator agent and binds to a g-protein coupled receptor Mas [7, 8]. Thus, ACE2 counterbalances Ang-II effects by decreasing the presence of this vasoconstrictor and by increasing the presence of a vasodilator (Ang(1-7)). These RAAS pathway proteins are present in high levels cardiovascular tissues including in the lung. Coincidentally the lung is the primary site of complications of respiratory infections from the corona-type viruses[8] including but not limited to SARS corona virus, and MERS corona virus.

Therefore, the RAAS pathway and associated lung tissue appear to by the mode of cellular entry of corona viruses including but not limited to COVID-19 virus.

### Definitions

The term “SNP” or “SNP” means single nucleotide polymorph. An SNP is a segment of a gene that displays variability in at least one and sometimes two nucleotide identities. Because of the SNP variations, the corresponding double stranded gene and its corresponding double stranded RNA sequence may be high functioning, moderately functioning, low functioning or not functional in terms of the activity of the corresponding protein.

The terms “homozygous” and “heterozygous” address the relationship between the strands of a double stranded DNA, cDNA, RNA and similar double stranded polynucleotides including their complementary SNPs. Homozygous means that the variable nucleotide at the polymorphic site is the same on both alleles of the SNP double stranded polynucleotide under consideration from an individual. Heterozygous means that the variable nucleotide at the polymorphic site on one allele differs from that on the other allele of the double stranded polynucleotide under consideration from an individual. The nucleotide variation may code for a highly functional peptide, a moderately functional peptide, a low functional peptide or a non-functional peptide.

The term “RAAS” means renin-angiotensin-aldosterone pathway and is the hormone system that regulates blood pressure, blood fluid and electrolyte balance and systemic vascular resistance.

The terms “ACE” and “ACE2” mean angiotensin converting enzyme and angiotensin converting enzyme 2. These enzymes are integral proteins located in the plasma membranes of certain kinds of cells such as but not limited to lung, kidney and vascular endothelial cells.

The term “renin” means an aspartic protease protein, also known as angiotensinogenase, is also a hormone and is secreted by the kidneys. It mediates the volume of blood plasma, lymph and interstitial fluids and mediates arterial vasoconstriction.

The terms “Ang-I”, “Ang-II”. “AT1 or AT1R” and “AT2 or AT2R” mean angiotensin I, angiotensin II, angiotensin II type 1 receptor and angiotensin II type 2 receptor respectively. The first two are renin and ACE enzymatic cleavage products derived from angiotensinogen. The last two are receptors for Ang-II.

The terms “Ang (1-9)” and “Ang (1-7)” mean angiogenin precursors (1-9) and (1-7) respectively and are the ACE2 hydrolysis products of Ang-I and Ang-II respectively.

The term “anti-viral pharmaceutical agent” means an organic small molecule or a small biomolecule designed to bind with one or more of a viral protein coat, viral RNA or with a cellular point of entry by a virus or a cellular membrane target site for a virus, or with viral RNA propagation or protein production.

The term “anti-bacterial agent” means an organic small molecule or a biological molecule that will interdict bacteria and cause one or more of apoptosis, lack of cellular division, tagging for antibody and/or immune response attack or another mechanism resulting in destruction of the bacteria.

As used herein, “obtaining a test sample” involves removing a sample of tissue from a patient, receiving a sample of tissue from a patient, receiving a patient's tissue sample from a physician, receiving a patient's tissue sample via mail delivery and/or removing a patient's tissue sample from a storage apparatus (e.g., a refrigerator or freezer) or a facility. Thus, obtaining a test sample can involve removal or receipt of the test sample directly from the patient, but obtaining a test sample can also include receipt of a test sample indirectly from a medical worker, from a storage apparatus/facility, from a mail delivery service after transportation from a medical facility, and any combination thereof. The test sample can therefore originate in one location and be transported to another location where it is received and tested. Any of these activities or combinations of activities involves “obtaining a test sample.”

As used herein a probe refers to a single DNA or RNA molecule (a nucleic acid oligomer) or a collection of nucleic acid molecules (nucleic acid oligomers) where the DNA molecules have at least one segment with a sequence that is complementary to a region of a target nucleic acid. The probe can hybridize with the target nucleic acid under stringent conditions. In some cases, the probe can hybridize with the target nucleic acid under highly stringent conditions. The probe is not identical to naturally available nucleic acids because has additional components such as one or more labels, one or more (engineered) restriction sites, one or more molecular barcodes, one or more tags for identification or retrieval of the probe (e.g., with or without the target hybridized thereto). In some instances, the probe is attached to a solid surface such as a chip, an array, a bead, or other surface.

As used herein a primer contains a region that is designed to hybridize to a targeted locus (e.g., a targeted polymorphic locus or a nonpolymorphic locus). The primer and may contain a priming sequence designed to allow PCR amplification. The primer can have at least one segment with a sequence that is complementary to a region of a target nucleic acid. The primer can hybridize with the target nucleic acid under stringent conditions. In some cases, the primer can hybridize with the target nucleic acid under highly stringent conditions. The primer is not identical to naturally available nucleic acids because has additional components such as a molecular barcode, a tag, an engineered restriction site, or a combination thereof. A primer may contain a random region that differs for each individual molecule. The terms “test primer” and “candidate primer” are not meant to be limiting and may refer to any of the primers disclosed herein.

As used herein a “binding entity” is a molecule or molecular complex that can recognize and bind to selected target molecules. Such binding entities can be antibodies or any molecule that has a binding domain for a target molecule.

Unless defined otherwise, all technical and scientific terms used herein have the same meaning as commonly understood by a person of ordinary skill in the art.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

All average molecular weights of polymers are weight-average molecular weights, unless otherwise specified.

The term “may” in the context of this application means “is permitted to” or “is able to” and is a synonym for the term “can.” The term “may” as used herein does not mean possibility or chance.

It is also to be understood that as used herein and in the appended claims, the singular forms “a,” “an,” and “the” include plural reference unless the context clearly dictates otherwise. The term “X and/or Y” means “X” or “Y” or both “X” and “Y”. The letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and the right is reserved to revise the application or claims to refer specifically to any individual member or any subgroup of members of the Markush group.

Single nucleotide polymorphisms for a number of genes are described herein. Further information about these genes and polymorphisms is available in the NCBI database at the following webpages:
ADRB1—rs1801252—ncbi.nlm.nih.gov/projects/SNP/snp_ref.cgi?rs=1801252
ADRB1—rs1801253—ncbi.nlm.nih.gov/projects/SNP/snp_ref.cgi?rs=1801253
ADRB2—rs1042713—ncbi.nlm.nih.gov/projects/SNP/snp_ref.cgi?rs=1042713
ADRB2—rs1042714—ncbi.nlm.nih.gov/projects/SNP/snp_ref.cgi?rs=1042714
WNK1—rs1159744—ncbi.nlm.nih.gov/projects/SNP/snp_ref.cgi?rs=1159744
WNK1—rs2106714—ncbi.nlm.nih.gov/projects/SNP/snp_ref.cgi?rs=2107614
WNK1—2277869—ncbi.nlm.nih.gov/projects/SNP/snp_ref.cgi?rs=2277869
Alpha adducin—rs4961—ncbi.nlm.nih.gov/projects/SNP/snp_ref.cgi?rs=4961
AGT—rs699—ncbi.nlm.nih.gov/projects/SNP/snp_ref.cgi?rs=699
AGT—rs7079—ncbi.nlm.nih.gov/projects/SNP/snp_ref.cgi.?rs=7079
AGT—rs4762 is a C to T substitution in AGT exon 2 with a consequent functional threonine (T) to methionine (M) exchange at codon 207 (termed T207M or T174M) (see ncbi.nlm.nih.gov/snp/?term=SNP+rs4762+)
AGT—rs5051—ncbi.nlm.nih.gov/projects/SNP/snp_ref.cgi?rs=5051
AGT1R or AT1R—rs5186—ncbi.nlm.nih.gov/projects/SNP/snp_ref.cgi?rs=5186
SLC12A3—rs1529927—ncbi.nlm.nih.gov/projects/SNP/snp_ref.cgi?rs=1529927
SCNN1A—rs2228576—ncbi.nlm.nih.gov/projects/SNP/snp_yef.cgi?rs=2228576
Renin—rs12750834—ncbi.nlm.nih.gov/projects/SNP/snp_ref.cgi?rs=12750834
ACE—rs1799752—ncbi.nlm.nih.gov/projects/SNP/snp_ref.cgi?rs=1799752
ACE2—rs112171234—ncbi.nlm.nih.gov/snp/rs112171234
ACE2—rs4646127—ncbi.nlm.nih.gov/snp/rs4646127

The following SNPs can also affect ACE2, for example, the function or activity of ACE2, or some other facet of the renin-angiotensin-aldosterone pathway:
rs12010448—ncbi.nlm.nih.gov/snp/rs12010448
rs143695310—ncbi.nlm.nih.gov/snp/rs143695310
rs1996225—nebi.nlm.nih.gov/snp/rs1996225
rs200781818 (ncbi.nlm.nih.gov/snp/?term=rs200781818—merged into rs60174128)
rs60174128—ncbi.nlm.nih.gov/snp/rs60174128
rs2158082—ncbi.nlm.nih.gov/snp/rs2158082
rs4060—ncbi.nlm.nih.gov/snp/rs4060
rs4830983—ncbi.nlm.nih.gov/snp/rs4830983
rs5936011—nebi.nlm.nih.gov/snp/rs5936011
rs5936029—nebi.nlm.nih.gov/snp/rs5936029
rs6629110—ncbi.nlm.nih.gov/snp/rs6629110

Pre and Post Infection of Coronaviruses Such as Coronavirus-19

The novel coronavirus (COVID-19) is believed to have evolved from human contact with animals such as bats. COVID-19 jumped the interspecies barrier and has resulted in severe respiratory ailments in humans similar to those of other coronaviruses including severe acute respiratory syndrome coronavirus (SARS-CoV) and MERS coronavirus. MERS coronavirus, COVID-19 and SARS-CoV bind to, and utilize, angiotensin-converting enzyme 2 (ACE2) as a host receptor in order to infect cells and cause respiratory illness in humans. ACE2 is expressed throughout the body, including in the lungs and vasculature, and the regulation of ACE2 is influenced by other factors in the RAAS pathway including renin. ACE, and the angiotensin receptors. Despite the rapidly increasing prevalence of COVID-19, and past history with SARS-CoV and MERS-CoV, it is clear that the infection in humans is heterogeneous. Some can carry and transmit the virus without any symptoms, i.e., are asymptomatic, some have mild symptoms, and some have severe enough complications resulting in death.

The two most common co-morbidities in patients who develop the most severe illness from COVID-19, and in those who have died from the virus, are hypertension and diabetes. Hypertension can cause an increase in endogenous ACE2 levels (as an inherent counter-regulatory pathway to balance increases in blood pressure) and treatment of hypertension with ACE-inhibitors and angiotensin-II receptor blockers can actually augment ACE2 levels. Patients with both type-I and type-II diabetes also have elevated ACE2 levels.

In regard to age related COVID-19, young humans can often transmit the disease without developing respiratory illness and men can be more dramatically affected than women. Although epidemiologists have primarily focused on behavior differences (smoking and drinking) between males and females, the data indicate that age and sex can influence endogenous ACE2 levels.

In animal model assessment of ACE2 levels, males often exhibit higher ACE2 expression and activity than females. This difference appears to be most prominent after puberty. Young animals often have far less ACE2 levels than older animals. In animal models, and in humans, reduced ACE2 and reduced levels of the product of ACE2 (angiotensin (1-7)) are associated with poor prognosis in patients with acute lung injury and acute respiratory distress syndrome.

Therefore, according to the invention, treatment of pre and post COVID-19 infection as well as infection by other coronaviruses are directed to identification of patients who have low vs. high ACE2 levels, based on genetic variants important in the renin-angiotensin-aldosterone system. Embodiments for treatment are directed to utilization of this genetic information to provide medication adjustments that can be utilized to reduce ACE2 concentrations prior to infection. Embodiments for treatment are also directed to identification of genetic groups that benefit from pharmacologic adjustments of the RAAS to increase ACE2 and ANG(1-7) levels after infection with a coronavirus.

Described herein are 21 SNPs (including those of beta-adrenergic receptors, angiotensinogen, renin, ACE, the type-I angiotensin receptor, and ACE2) that are important in the endogenous modulation of ACE2. The genotype of a person or patient with respect to the different types of SNPs can guide medication adjustments for decreasing or increasing the expression, function and activity of ACE2.

According to the present invention, the data regarding asymptomatic persons, patients who exhibit mild symptoms, and those who have developed severe respiratory complications, including acute respiratory distress syndrome and death indicate that investigation of differences in endogenous ACE2 levels will enable alteration of the risk of disease transmission and will enable alteration of the degree of severity of disease progression. Pursuant to the concept according to the invention involving COVID-19's and other coronavirus's targeting ACE2 present in membranes of lung epithelial cells, embodiments of the method for treatment according to the invention depend upon the status of the person under consideration for treatment and interdicting negatively or positively the level of ACE2.

If the person has been or could have been exposed to COVID-19 but is not yet infected by COVID-19 or another coronavirus, the person is administered RAAS pharmaceutical agents designed to reduce the level of ACE2 in the person, especially in the lung pathway cells. One exemplary pharmaceutical agent for lowering ACE2 levels in humans is a renin inhibitor. Although it is not a limitation of the invention, it is believed that minimizing or shutting down the ACE2 levels in such a person will deny to COVID-19 or other coronavirus the ability to gain cellular entry and COVID-19 or other coronavirus reproduction. It is also recognized that full shutdown without adverse complications is likely so that full minimization of ACE2 levels is a desired outcome. It is believed that denial of cellular entry for COVID-19 or other coronavirus will enable the person's functions such as mucus, cilia and similar respiratory ejection functions to eliminate the COVID-19 particles or other coronavirus particles and/or to metabolically and/or immunogenically eliminate the COVID-19 particles or other coronavirus particles from the person. Determination of the susceptibility of the person to ACE2 reduction depends upon certain combinations of 21 SNPs associated with the person's RAAS pathway.

In contrast, if the person has been exposed to COVID-19 or other coronavirus and tests positive for the presence of COVID-19 or other coronavirus and is either asymptomatic or exhibits symptoms of COVID-19 or other coronavirus infection so that the person is now a patient, the patient is administered RAAS pharmaceutical agents designed to increase the level of ACE2. Exemplary pharmaceutical agents suitable for increasing ACE2 levels in humans include ACE inhibitors. ATiR blocker, AT2R blocker (ARB's) and beta blockers. Although it is not a limitation of the invention, it is believed that with COVID-19 or other coronavirus infection, COVID-19 or other coronavirus coopts ACE2 function. Because ACE and ACE2 are counter-veiling enzymes of the RAAS pathway, co-opting, negating and/or minimizing of ACE2 function is believed to shut down and/or minimize the lung and cardiovascular protective effects of ACE2. Use of RAAS pharmaceutical agents designed to boost ACE2 production is believed to re-ignite the protective effects of ACE2 and prevent and/or ameliorate adverse lung and/or cardiovascular events associated with symptomatic infection by COVID-19 or other coronaviruses.

According to embodiments of the invention, choice of an RAAS pharmaceutical agent for pre-infection persons and for post-infection patients may be made according to the person or patient's 21 SNPs associated with the genes coding for enzymes that can affect ACE and other RAAS functions, including angiotensinogen (AGT), renin (REN), angiotensin converting enzyme (ACE), angiotensin II receptor type 1 (AT1R), angiotensin II receptor type 2 (AT2R), angiotensin converting enzyme 2 (ACE2), and adrenergic receptor beta 1 (ADRB1). These genes are part of the RAAS pathway or have an effect on the RAAS as is the case with ADRB1. One or more combinations of these SNPs are responsible for enabling levels of ACE2 that are a) highly functional, b) moderately functional, c) minimally functional and/or d) not functional. In other words, the expression, function and/or activity of ACE2 is affected by such combinations. These categories a-d depend upon the importance of individual SNPs upon ACE2 levels. Because the combinations of the 21 SNPs together result in overall influence of increase and/or decrease of ACE2 levels, certain combinations enable determination of this influence and indicate the kind of RAAS pharmaceutical agent for producing the desired decrease or increase of ACE2 levels respectively in the person or patient.

The SNPs exist as genotype alleles for the genes involved. Because the genes have paired nucleotide sequences, the genotype alleles are paired sequences. These alleles may be homozygous for the desired SNP having a positive influence on the protein expression of the gene involved. The homozygous arrangement means that both SNPs of the paired sequence have the positive influence. The alleles may be heterozygous for the desired SNP having a positive influence on the protein expression of the gene involved. The heterozygous arrangement means that one of the SNP pairs has a positive influence and the other has a minimal influence or a null influence on protein expression. The alleles may also be homozygous for the undesired SNP so that both SNPs have either minimal influence or a null influence on protein expression.

Each desired SNP has a positive rating factor ranging from 1 to 10, with 10 being the highest rating, for influence of level of protein expression by the corresponding gene. The corresponding undesired SNP may have a moderate effect, a null effect or a minimal effect upon the level of protein expression. For homozygous alleles with desired SNPs and heterozygous alleles the rating will be a combination of those of the individual SNPs. For homozygous alleles with undesired SNPs the rating will also be a combination of those of the individual SNPs. The heterozygous combination can act as fully-functional (giving a weighted score of 10), partially functional (giving a weighted score of 5), or marginally or non-functional, i.e., null functional (giving a weighted score of 1).

With the foregoing understanding of the inter-relationship of the SNPs their alleles and the RAAS genes involved in the RAAS pathway, a person not yet infected with COVID-19 or other coronavirus may be treated to avoid and/or minimize COVID-19 or other coronavirus infection by lowering the person's ACE2 level through administration to the person of a pharmaceutical compound or compounds according to the following pre-infection protocols.

Pre-infection Protocol 1 provides that the SNPs of this genotype are a particular combination of SNP rs numbers with the variable nucleotides indicated in parenthesis including:
a) angiotensinogen (AGT) SNPs including rs5051(T), rs699(C) are positive homozygous alleles or positive heterozygous alleles; and rs4762(T) and rs7079(G) are only positive as homozygous alleles;
b) renin (REN) SNP having rs12750834(A) is positive as homozygous alleles or as heterozygous alleles;
c) angiotensin-converting enzyme (ACE) SNP having rs1799752(Deletion) is positive as homozygous alleles or heterozygous alleles;
d) angiotensin converting enzyme 2 (ACE2)-related SNPs include any three or more positive homozygous alleles selected from the group of rs112171234(G), rs12010448(C), rs143695310(A), rs1996225(T), rs200781818(ins), rs2158082(A), rs4060(A), rs4646127(G), rs4830983(C), rs5936011(T), rs5936029(T), and rs6629110(C);
e) angiotensin receptor type-1 (AT1R) SNP having rs5186(C) positive homozygous alleles, and;
f) the beta-1 adrenergic receptor (ADRB1) SNP rs1801252(A) or SNP rs1801253(C) wherein the alleles of both are positive in homozygous or heterozygous form.

Pre-infection Protocol 1 provides that the pharmaceutical compounds to be administered include a renin inhibitor and/or beta blocker and that the administration of an ACE inhibitor and an angiotensin receptor blocker (ARB) are to be discontinued if the person has been receiving either or both of the ACE inhibitor and/or the ARB. The SNPs of Protocol 1 genotype indicate a high functionality in production and/or activity of the following enzymes: alanine-glyoxylate transaminase (AGT), rennin (REN), angiotensin converting enzyme (ACE) and angiotensin converting enzyme 2 (ACE2).

Pre-infection Protocol 2 provides that the SNPs of this genotype are a particular combination of SNP rs numbers with the variable nucleotides indicated in parenthesis, including:
a) REN SNP rs12750834(A) as positive homozygous alleles or as heterozygous alleles,
b) AT1R SNP rs5186(C) as positive homozygous alleles;
c) ADRB1 SNP rs1801252(A) and rs1801253(C) as positive homozygous alleles or heterozygous alleles, and;
d) the AGT, ACE, ACE2, and ADRB1 SNPs have no positive homozygous or heterozygous alleles.

Pre-infection Protocol 2 provides that the pharmaceutical compounds to be administered are a renin inhibitor and/or beta blocker and administration of an ARB is to be discontinued if the person has been receiving the ARB. The SNPs of Protocol 2 genotype indicate a high functionality in REN and adrenergic receptor beta 1 (ADRB1), a low functionality in AT1a and no functionality in AGT.

Pre-infection Protocol 3 provides that the SNPs of this genotype are a particular combination of SNP rs numbers with the variable nucleotides indicated in parenthesis, including:
a) AGT SNPs rs5051(T), rs699(C) as positive homozygous alleles or heterozygous alleles, rs4762(T) and rs7079(G);
b) REN SNP rs12750834(A) as positive homozygous alleles or heterozygous alleles;
c) AT1R SNP rs5186(C) as positive homozygous alleles;
d) and the ACE SNPs have no positive homozygous alleles or heterozygous alleles.

Pre-infection Protocol 3 provides that the pharmaceutical compounds to be administered are a renin inhibitor and/or a beta blocker and/or an ACE inhibitor and the administration of an ARB is to be discontinued if the person has been receiving the ARB. The SNPs of Protocol 3 genotype indicate a moderate to low functionality in AGT, REN, AT1a and ADRB1 and no functionality in AGT.

Pre-infection Protocol 4 provides that the SNPs of this genotype are a particular combination of SNP rs numbers with the variable nucleotides indicated in parenthesis, including:
a) ACE SNP rs1799752(Deletion) as positive homozygous alleles or heterozygous alleles and D indicates a deletion.

Pre-infection Protocol 4 provides that and no pharmaceutical compound is to be administered and administration of an ACE inhibitor and/or an ARB are to be discontinued if the patient has been receiving either or both of the ACE inhibitor and/or the ARB. The SNPs of the Protocol 4 genotype indicate high functionality in ACE and no functionality in ACT, REN and ABRB1.

Pre-infection Protocol 5 provides that the products of the weight numbers times the influence numbers for all 21SNPs of a person's genotype are to be summed together. The weight numbers and influence numbers are found for each individual positive homozygous SNP allele, heterozygous SNP allele and/or null homozygous SNP allele as provided in the following section and its Table 1. If the sum of these products of the 21 SNPs listed on Table 1 is greater than 400, Pre-infection Protocol 5 provides that the pharmaceutical compound to be administered is a renin inhibitor, Protocol 5 further provides that if the person has been administered any or all of ACE inhibitors and/or ARBs continued administration of these pharmaceutical compounds should be discontinued.

Pre-infection Protocol 6 provides that if the 21 SNPs constituting the genotype of the person do not present the SNPs of Protocols 1-4 and the sum of the products of the weight and influence numbers for these genotypes do not sum to greater than 400 as provided in Protocol 5, then no administration of a pharmaceutical compound for lowering the person's ACE2 level is made. This person already has low or practically no functional ACE2 levels.

With the foregoing understanding of the inter-relationship of the SNPs, their alleles and the RAAS genes involved in the RAAS pathway, a patient who is infected with a coronavirus such as but not limited to COVID-19 may be treated to ameliorate, decrease and/or otherwise minimize the coronavirus or COVID-19 infection by increasing the patient's ACE2 level and Ang(1-7) level through administration to the patient of a pharmaceutical compound or compounds according to the following protocols. For the post-infection protocols the variable nucleotide of the designated SNP may provide a low or null functional allele or a positive functional allele depending upon whether the gene and protein corresponding to the SNP has positive influence on the expression, function and activity of ACE2 or has null influence. For example, an ACE2 SNP allele with low or null function with respect to expression, function and activity of ACE2 protein would have null influence on ACE2 and would indicate that the ACE2 level should be boosted. This contrasts with the pre-infection protocols which look for the high or positive functional alleles. Post-infection analysis determines whether the expression, function and activity of ACE2 is low and needs to be boosted. The 21 SNPs of a patient's genotype that have a reductive or suppressive influence in this regard are to be determined (e.g., an ACE SNP allele that is positive will enable a suppressive influence on ACE2 levels).

Post-infection Protocol 1 provides that the SNPs of this genotype are a particular combination of SNP rs numbers with the variable nucleotides indicated in parenthesis, including
a) AGT SNPs rs5051(A) low functional or null homozygous alleles or heterozygous alleles, rs699(G) only low or null functional homozygous alleles, rs4762(A) and rs7079(C) both of which may be low or null functional homozygous alleles or heterozygous alleles;
b) REN SNP rs12750834(T) low or null functional homozygous alleles only;
c) AT1R SNP rs5186(T) low or null functional homozygous alleles or heterozygous alleles;
d) ACE2 SNPs including any three or more low or null functional homozygous alleles selected from rs112171234(C), rs12010448(G), rs143695310(T), rs1996225(A), rs200781818(Deletion), rs2158082(T), rs4060(T), rs4646127(C), rs4830983(G), rs5936011(A), rs5936029(A), and rs6629110(G);
e) ADRB1 SNP either as rs1801252(T) or rs1801253(G) as low or null functional alleles only, and;
f) ACE SNP as rs1799752(Deletion) as positive homozygous alleles or as heterozygous alleles.

Post-infection Protocol 1 provides that the pharmaceutical compounds to be administered are an ACE inhibitor and/or ARB. Administration of these compounds boost the patient's production of ACE2 and Ang(1-7) levels for a patient with this genotype. The SNPs for the post-infection Protocol 1 genotype indicate a low functionality in AGT, REN, AT1a and ADRB1 and high functionality in ACE. Consequently, the patient's ACE2 levels will be boosted though administration of RAAS pharmaceuticals that affect the dynamic equilibrium of ACE and ACE2.

Post-infection Protocol 2 provides that the SNPs of the genotype are a particular combination of SNP rs numbers with the variable nucleotides indicated in parenthesis, including:
a) REN SNP rs12750834(A) positive homozygous alleles or heterozygous alleles;
b) ADRB1 SNPs rs1801252(A) and rs1801253(C) as positive homozygous alleles;
c) AT1R SNP rs5186(C) as positive homozygous alleles;
d) AGT, ACE2, and ACE SNPs as heterozygous alleles.

Post-infection Protocol 2 provides that the pharmaceutical compounds administered an ARB and/or a beta blocker. Administration of these compounds boost the patient's production of ACE2 and Ang(1-7) levels for a patient with this genotype. The SNPs for the Post-infection Protocol 2 genotype indicate a high functionality in REN and ADRB1, a functionality in AT1a and no functionality in AGT and ACE. Consequently, the patient's ACE2 levels will be boosted through administration of RAAS pharmaceuticals that will affect renin and ADRB1 levels.

Post-infection Protocol 3 provides that the SNPs of the genotype are a particular combination of SNP rs numbers with the variable nucleotides indicated in parenthesis, including:
a) AGT SNP rs5051(T) as positive homozygous alleles, rs699(C) as positive homozygous alleles or heterozygous alleles, rs4762(T) and rs7079(G) both as positive homozygous alleles;
b) REN SNP rs12750834(A) as positive homozygous alleles or heterozygous alleles;
c) AT1R SNP rs5186(A) as positive homozygous alleles;
d) ADRB1 SNP either as (rs1801252(T) or rs1801253(G) as low or null homozygous alleles, and;
e) ACE and ACE2 SNPs as heterozygous alleles.

Post-infection Protocol 3 provides that the pharmaceutical compound administered is an ARB. Administration of this compound boost the patient's production of ACE2 and Ang(1-7) levels for a patient with this genotype. The SNPs for Post-infection Protocol 3 genotype indicate a high functionality in ADRB1, REN and AT1a but moderate to minimal functionality in AGT, ACE2 and ACE. Consequently, the patient's ACE2 levels will be boosted through administration of RAAS pharmaceuticals that will affect renin, ADRB1 and AT1a levels.

Post-infection Protocol 4 provides that the SNPs of the genotype are a particular combination of SNP rs numbers with the variable nucleotides indicated in parenthesis, including:
a) ACE SNP rs1799752(Deletion) as positive homozygous alleles or heterozygous alleles;
b) AT1a SNP rs5186(A) as positive homozygous alleles;
c) ACE2 SNPs including any three or more low or null homozygous alleles selected from rs112171234(C), rs12010448(G), rs143695310(T), rs1996225(A), rs200781818(Deletion), rs2158082(T), rs4060(T), rs4646127(C), rs4830983(G), rs5936011(A), rs5936029(A) and rs6629110(G), and;
d) AGT, REN, or ADRB1 SNPs as heterozygous alleles.

Post-infection Protocol 4 provides that the pharmaceutical compounds to be administered are an ACE inhibitor and/or an ARB. Administration of these compounds boost the patient's production of ACE2 and Ang(1-7) levels for a patient with this genotype. The SNPs for Post-infection Protocol 4 genotype indicate high functionality in ACE and AT1a but low or null functionality in AGT, REN and ABRB1. Consequently, the patient's ACE2 levels will be boosted through administration of RAAS pharmaceuticals that will affect ACE and AT1a levels.

Post-infection Protocol 5 provides that the products of the weight numbers times the influence numbers for all 21SNPs of a patient's genotype are to be summed together. The positive homozygous allele, heterozygous allele and null homozygous weight numbers and influence numbers for each of the individually determined SNPs are found as provided in the following section and its Table 2. If the sum of these products of the 21 SNPs listed on Table 2 is less than 550, Protocol 5 provides that the pharmaceutical compounds to be administered are one or more of ACE inhibitors and/or ARB's and/or beta blockers.

Post-infection Protocol 6 provides that if the genotype of the patient does not present the SNPs of Protocols 1-5, no administration of a pharmaceutical compound for increasing the patient's ACE2 and Ang(107) levels are made. This patient already has ACE2 levels at a high functional level.

For all Post-infection Protocols 1-5, agonists for Ang (1-7), the co-protein and product of ACE2 hydrolysis of Ang-II may be administered to boost the cardiovascular protective effect of ACE2 and Ang(1-7). Exemplary agonists include a nonapeptide angiotensin (1-7) receptor Mas agonist of the formula Asp¹-Arg²-Val³-Ser⁴-Ile⁵-His⁶-Cys⁷ and the formula Asp¹-Arg²-Val³-Tyr⁴-Ile⁵-His⁶-Pro⁷ as well as a non-peptide compound having the chemical formula 5-formyl-4-methoxy-2-phenyl-1[[4-[2-ethylaminocarbonylsulfonamido)-5-isobutyl-3-thienyl]phenyl]methyl]imidazole. See for example U.S. Pat. No. 8,383,772.

The foregoing Pre-infection and Post-infection Protocols categorize certain segments of the human population according to groupings of the 21 SNPs having certain arrangements of positive homozygous alleles, heterozygous alleles and null homozygous alleles. Other segments of the human population are included according to the procedures outlined Pre-infection and Post-infection Protocols 5 and 6. In particular, Pre-infection and Post-infection Protocols 5 are determined by summing the individual SNP weight numbers and individual SNP influence numbers according to the following explanations and procedures.

SNP Weight and Influence Scores for Calculation of Combinatorial Results for Pre- and Post-Infection Protocols 5

The RAAS pathway manages a number of physiological activities in humans, including but not limited to blood pressure, fluid and electrolyte balance and systemic vascular resistance of the cardiovascular and lymph systems. Control of the RAAS pathway depends upon the interactions and endogenous levels of a number of proteins intersecting and/or directly functioning within the RAAS pathway. These proteins (and associated genes) include but are not limited to angiotensinogen (AGT gene), renin (REN gene), angiotensin converting enzyme (ACE gene), angiotensin converting enzyme 2 (ACE2 gene), angiotensin I (Ang-I gene), angiotensin II (Ang-II gene), angiogenin precursor (1-9) (Ang (1-9) gene), angiogenin precursor (1-7) (Ang(1-7) gene), angiotensin II type 1 receptor (AT1R gene), angiotensin II type 2 receptor (AT2R gene), beta-1 adrenergic receptor (ADRB1 gene), beta-2 adrenergic receptor (ADRB2 gene), epithelial sodium channel protein (SCNN1A gene), alpha-adducin (ADD1 gene), sodium chloride cotransporter (SLC12Ae gene), lysine deficient protein kinase-1 (WNK1 gene), as well as other proteins and corresponding genes. According to aspects of the invention, the genes and proteins of the RAAS pathway that exhibit dynamic interactions leading to the expression, function and activity of ACE2 are AGT, renin, AT1R, ACE, ADRB1 and of course, the ACE2 gene and protein themselves.

Coronaviruses are a group of related, common viral agents responsible for disease in mammals and birds. In humans, coronaviruses are responsible at least in part for the common cold as well as other mild illnesses. More serious infections are caused by such corona virus species as Mers coronavirus SARS coronavirus and covid-19 virus among others. The coronavirus generally is known to target the ACE2 protein present as integral constituent of the membranes of lung epithelial cells. This target provides a means for multiple species of coronavirus to gain cellular entry into human hosts. Therefore, the present invention is directed to control of the level of ACE2 in human hosts in order to lessen the ability of coronavirus to invade and infect human hosts.

Because the ACE2 protein is part of the RAAS pathway and is dynamically interactive with a number of other RAAS proteins, the present invention is directed to control of the ACE2 protein expression, function and activity through management of the RAAS proteins that dynamically interact with ACE2 as well as through direct intercession of ACE2 levels. According to the present invention, the RAAS proteins and genes in dynamic interaction with ACE2 include AGT, renin, ACE, AT1R, ACE2, ADRB1. The pharmacological and physiological interactions of these proteins/genes manages the dynamic expression, function and activity of ACE2.

The genes corresponding to these interactive RAAS proteins for ACT, renin, ACE. AT1R, ACE2 and ADRB1 exhibit known polymorphisms which are common and ordinary across the human population. The SNPs containing the nucleotide variations providing the polymorphisms of these genes include:
a) for AGT, rs5051, rs699, rs4762 and rs7079;
b) for renin, rs12750834;
c) for ACE, rs1799752;
d) for AT1R, rs5186;
e) for ACE2, rs112171234, rs12010448, rs143695310, rs1996225, rs200781818, rs2158082, rs4060, rs4646127, rs4830983, rs5936011, rs5936029, and rs6629110;
f) for ADRB1, rs1801252 and rs1801253.

The double stranded SNPs presented by these genes are alleles in that the nucleotide variation providing the polymorphism of the SNPs may be the same or may be different according to complementary pairing. If both segments contain the same nucleotide at this polymorphism site, they are homozygous. If the two segments contain complimentary nucleotides at the polymorphism site, they are heterozygous. The corresponding single strand SNP within the mRNA downstream of the DNA allele will carry one of these two variations. Hence, the corresponding protein in humans may have a variable amino acid group corresponding to this SNP polymorphism. The prevalence of the polymorphism in the human population, means that segments of the population will exhibit the polymorphism variations in their corresponding expressed proteins.

According to the invention, it has been discovered that some polymorphisms expressed in the AGT, renin, ACE, AT1R, ACE2 and ADRB1 proteins lead to significant enhancement of the expression, function and activity of the ACE2 protein while other polymorphisms lead to moderate enhancement of the expression, function and activity of the ACE2 protein and still other polymorphisms lead to null or neutral expression, function and activity of the ACE2 protein. According to the invention, it has been discovered that the expression, function and activity of the ACE2 protein influences the ability of the coronavirus particle to invade host cells having the ACE2 protein on their membranes. Low expression, function and/or activity of ACE2 leads to lower virus invasion and correspondingly lower infection according to aspects of the present invention.

In particular according to aspects of the invention, the homozygous or heterozygous character of the alleles coding the foregoing SNPs leads to and influences greater or lesser enhancement of the expression, function and activity of the ACE2 protein. The influence depends upon which polymorphism of the allele leads to enhancement and which does not. For a homozygous allele in which both segments contain the influential polymorphism, the resulting enhancement of the expression, function and activity of the ACE2 protein can be significant. This SNP allele is a positive homozygous allele. For a heterozygous allele having one segment with the influential polymorphism, the enhancement may or may not be significant depending upon which of the SNP segments is transcribed, translated and expressed and the enhancement balance between the differing segments of the allele. It also depends upon whether or not the other segment displays some or no influence. For a homozygous allele in which both segments contain the lesser influential or non-influential polymorphism, the resulting expression, function and activity of the ACE2 protein can be minimal. This SNP allele is a null homozygous allele.

Because all of the foregoing SNPs have a bearing directly or indirectly upon the expression, function and activity of the ACE2 protein and because their translation, transcription and expression are subject to feed-back biological mechanisms, the assessment of the influence of the foregoing SNPs is subject to biological complexities. According to the invention, these complexities have been addressed through evaluation of the medical condition and genetic make-up of patients suffering from coronavirus infections such as the MERS virus, the SARS virus as well as the Covid-19 virus. This information has enabled development of individual weighting of the foregoing individual SNPs in regard to the likelihood of their influence on ACE2 expression, function and activity (e.g., ACE2 levels in cells).

A high weighting number is given for the SNP polymorphism producing the most influence on ACE2 levels. The individual nucleotides of the variable nucleotide site of the SNP polymorphisms for AGT, renin, ACE, AT1R, ACE2 and ADRB1 that exhibit positive influence on ACE2 expression are provided in parenthesis following each rs number. The terms Deletion and Ins that are marked with an asterisk mean deletion and insertion of a repeat nucleotide. The null influence SNP has the complement nucleotide at the variable nucleotide site of the SNP, e.g., A and T or C and G. The SNP positive weight polymorphisms are:
a) for AGT, rs5051(T), rs699(C), rs4762(T) and rs7079(G);
b) for renin, rs12750834(A);
c) for ACE, rs1799752(Deletion*);
d) for AT1R, rs5186(C);
e) for ACE2, rs112171234(G), rs12010448(C), rs143695310(A), rs1996225(T), rs200781818(ins*), rs2158082(A), rs4060(A), rs4646127(G), rs4830983(C), rs5936011(T), rs5936029(T), and rs6629110(C);
f) for ADRB1, rs1801252(A) and rs1801253(C).

The RAAS system and its many regulatory proteins are in dynamic balance and this balance affects and manages the outcomes of the RAAS system. Consequently, no one single homozygous or heterozygous SNP will be responsible for positive influence or null-influence in regard to expression, function and activity of ACE2. Persons in pre-infection situations and patients in post-infection situations will have combinations of the homozygous and heterozygous alleles for the SNPs relating to AGT, renin, ACE, AT1R, ACE2 and ADRB1 proteins. These combinations in sum are responsible for the degree of influence over the expression, function and activity of ACE2. Therefore, according to the invention, the overall assessment of a pre-infection person or a post-infection person will also depend upon the person or patient's genotype combination of these 21 SNPs that produce significant influence, produce moderate influence, produce low influence or produce no influence. This combination is determined according to the invention through use of an influence score associated with each SNP. The influence score rates the influence of the SNP in terms of the role and influence its corresponding gene and protein have upon the RAAS pathway. The genotype contribution of an SNP is determining by multiplying its weight score times its influence score. These scores are provided on Tables 1 and 2.

For pre-infection, the individual weight scores of the SNPs have been determined according to the invention through survey of historical genotype data and information regarding the individual SNPs and their impact on the transcription, translation and expression of the AGT, renin. ACE, AT1R, ACE2 and ADRB1 genes and proteins. The individual weight scores for the SNPs of a person with a pre-infection condition are given in Table 1 under the column headings Positive Homoz Fx, Heteroz Fx and Null Fx. These scores range from 1-10 with 10 being highest.

If the allele for an SNP is positive homozygous meaning both SNP segments contain the same positive influence nucleotide at the variable nucleotide site, the weight score is always 10. This score is indicated on the Positive Homoz Fx column of Table 1. If the allele for an SNP is null homozygous meaning both SNP segments contain the same null influence nucleotide at the variable nucleotide site, the weight score is always 1. The scores for the null weight are listed on Table 1 in the Null Fx column. If the allele for an SNP is heterozygous meaning that one SNP segment contains the positive influence nucleotide and the other contains the null influence nucleotide, the weight of the heterozygous allele varies as indicated on the Hetero Fx column of Table 1. The heterozygous weight was developed according the invention through a survey and review of data and information about the SNP heterozygous allele activities in the RAAS pathway.

These pre-infection individual weight scores are for the SNPs of alleles that impact the ACE2 levels and are shown individually on Table 1. As a theoretic example, for the renin SNP rs12750834, the variable nucleotide is A/T. The renin SNP that produces significant influence on ACE2 levels is the renin SNP with A. In contrast, the renin SNP with T produces low to null influence on ACE2 levels. Consequently, a homozygous renin allele with A as the variable nucleotide has a weight score of 10, see the renin row, Homoz Fx column of Table 1 which lists a score of 10. Because renin exhibits significant impact on the RAAS pathway and upon ACE2, the heterozygous renin SNP also has a weight score of 10, see the renin row, Heteroz Fx column of Table 1 which lists a score of 10. Other heterozygous SNPs of the list may not have such a significant impact so that their weight scores are 5, see the Heteroz Fx column of Table 1. In contrast, a homozygous renin allele with T as the variable nucleotide will produce little or no impact, see the renin row, Null Fx column of Table 1 which lists a score of 1.

In addition to the individual weight scores for the individual homozygous and heterozygous SNPs, the corresponding proteins exert greater and/or lesser overall influence over the management and function of the RAAS pathway irrespective of their polymorphic amino acid variations. This functional influence also affects the expression, function and activity of the ACE2 protein. According to the invention, the functional influences of the proteins of the RAAS pathway also relate back to evaluation of impact of the individual SNPs upon the expression, function and activity of ACE2. For pre-infection, the functional importance of these SNPs for their likelihood of impacting endogenous ACE levels is scored also on a scale of 1-10 with 10 being highest. The importance scores do not address the positive or null homozygous or heterozygous configurations of the SNPs but rather account for the role of the corresponding gene and protein in the RAAS pathway. These importance scores for pre-infection are:
1) highest functioning with an importance score of 10—Renin, rs12750834; ACE, rs1799752. AT1R, rs5186, ACE2 (rs2158082, rs4060, rs4646127, rs4830983, rs5936011, rs5936029, rs6629110);
2) moderately high functioning with a weight score of 8—AGT (rs699), ACE2 (rs112171234, rs12010448, rs143695310, rs1996225, rs200781818, rs2158082, rs4060, rs4646127, rs4830983);
3) moderate functioning with a weight score of 7—AGT (4762) and ADRB1 (rs1801253);
4) low moderate functioning with a weight score of 6—AGT (rs5051 and rs7079) and ADRB1 (rs1801252).

For post-infection, the individual weight scores of the SNPs have been determined according to the invention through survey of historical genotype data and information regarding the individual SNPs and their impact of AGT, Renin, ACE, AT1R, ACE2 and ADRB2 genes and proteins. The individual weight scores for the SNPs of a patient with a post-infection condition are given in Table 2 under the column headings Positive Homoz Fx, Hetero Fx, and Null Fx. These scores range from 1-10 with 10 being highest.

If the allele for an SNP is positive homozygous meaning both SNP segments contain the same positive influence nucleotide at the variable nucleotide site, the weight score is always 10 as indicated on the Positive Homoz Fx column of Table 2. If the allele for an SNP is null homozygous meaning both SNP segments contain the same null influence nucleotide at the variable nucleotide site, the weight score is always 1 as indicated on the Null Fx column of Table 2. If the allele for an SNP is heterozygous meaning that one SNP segment contains the positive influence nucleotide and the other contains the null influence nucleotide, the weight of the heterozygous allele varies as indicated on the Hetero Fx column of Table 2. The heterozygous weight was developed according the invention through a survey and review of data and information about the SNP heterozygous allele activities in the RAAS pathway.

In addition to the individual weight scores for the individual homozygous and heterozygous SNPs, the corresponding proteins exert greater and/or lesser overall influence over the management and function of the RAAS pathway irrespective of their polymorphic amino acid variations. This functional influence also affects the expression, function and activity of the ACE2 protein. According to the invention, the functional influence of the proteins of the RAAS pathway also relates back to evaluation of impact of the individual SNPs upon the expression, function and activity of ACE2. For post-infection, the functional importance of these SNPs for their likelihood of impacting endogenous ACE levels are scored also on a scale of 1-10 with 10 being highest. The importance scores do not address the positive or null homozygous or heterozygous configurations of the SNPs but rather account for the role of the corresponding gene and protein in the RAAS pathway. These importance scores for post-infection are:
1) highest with an importance score of 10—ACE, rs1799752; AT1R, rs5186;
2) moderately high with an importance score of 9—REN (rs12750834) and AGT (rs699);
3) moderate with an importance score of 8—ACT (rs4762), ACE2 (rs2158082, rs4060, rs4646127, rs4830983, rs5936011, rs5936029, rs6629110);
4) low moderate with an importance score of 7—AGT (rs7079 and rs5051) and ADRB1 (rs1801253);
5) low with an importance score of 6—ADRB1 (rs1801252) and ACE2 (rs112171234, rs12010448, rs143695310, rs1996225, rs200781818, rs2158082, rs4060, rs4646127, and rs4830983).

The weight scores and influence scores indicate the degree of impact of the SNPs their alleles upon ACE2 levels. However, for the post-infection situation, an aspect of the invention provides that the patient who suffers for serious infection issues has a LOW ACE2 level. This aspect of the invention holds that the low level of the ACE2 should be increased to counterbalance the other regulatory proteins of RAAS and protect the respiratory, cardiovascular and kidney systems from the adverse effects of coronavirus infection. The infection co-opts ACE2 so that it no longer provides its ordinary counterbalance in the RAAS system.

Consequently, patients infected with coronavirus and who have SNP alleles indicating little or no influence in regard to ACE2 expression, function and activity are in need of a boost of endogenous ACE2. For this reason, the protocols according to the invention for post-infection address the patients having SNPs producing low ACE2 functionality. As a theoretic example, a patient with AGT(rs5051) with the variable nucleotide as A, AGT(rs669) with the variable nucleotide as G, and AGT(rs7079) with the variable nucleotide as C theoretically will have SNPs producing practically little influence over the expression, function and activity of ACE2. A theoretic protocol for this patient would be to administer an ACE inhibitor and an ARB to boost production of ACE2.

The RAAS system and its many regulatory proteins are in dynamic balance which affects and manages the outcomes of the RAAS system. As mentioned above, no one single homozygous or heterozygous SNP will be responsible for positive influence or null-influence in regard to expression, function and activity of ACE2. Persons in pre-infection situations and patients in post-infection situations will have combinations of the homozygous and heterozygous alleles for the SNPs relating to AGT, renin, ACE, AT1R, ACE2 and ADRB1 proteins. According to the invention, the overall assessment of ACE2 level influence is made by addressing a person or patient's genotype constituting a combination of the 21 homozygous positive, homozygous null and heterozygous alleles in terms of the weight scores and the influence scores.

To determine how the combinations of the 21 SNPs interact for pre-infection persons and post-infection patients, the 21 SNP sequences of the persons or patients are assessed to identify their homozygous positive, heterozygous or homozygous null status of these 21 SNPs. Depending upon the homozygous or heterozygous status, these particular alleles of the person's or patient's genotype receive weight scores.

The SNP weight score is determined according to the functional genotype and the HomoZ FX and Heteroz Fx score columns of Table 1 (pre-infection) and Table 2 (post-infection). If an individual has an SNP that is homozygous for the positive polymorphism (two functional alleles) then that SNP receives a score of 10. Similarly, if an individual has an SNP that is heterozygous, then that SNP receives the score indicated in the Heteroz Fx column score.

Following assignment of the individual SNP weight scores for the 21 SNPs of a pre-infection person or a post-infection patient, the importance score for each gene SNP is taken from the Importance column of Tables 1 and 2. The importance score is independent of the homozygous or heterozygous character of the allele. The importance score is a relative value indicating the degree of influence the proteins, the genes, their SNPs have over the expression, function and activity of ACE2 protein in the contexts of pre-infection and post-infection. These importance scores were determined by assessment of literature reports of the genotype and SNPs of patients and their RAAS functions, who were infected by coronaviruses such as the SARS virus, and assessments of these aspects relative to the severity of the infections. The importance scores for pre- and post-infection for a particular SNP allele differ.

The combinatorial score for a pre-infection person or a post-infection patient is obtained from the combination of the SNP weight scores and the importance scores according to the person or patient's particular genotype for the 21 SNPs listed above. For pre- and post-infection persons and patients respectively each individual SNP weight score is multiplied by the importance score for each SNP of the genotype and the results of the 21 multiplications for all 21 SNPs are summed.

The sum providing the combinatorial score indicates whether a pharmaceutical compound should be administered and/or withdrawn. The choice depends upon whether the subject is a person with a pre-infection condition, or the subject is a patient with a post-infection condition. According to the invention for the pre-infection condition, the ACE2 level should be minimized as much as possible. For the post-infection condition, the ACE2 level should be maximized as much as possible. Choice of administration and/or withdrawal depends upon these opposing conditions and upon the kind of combination score obtained.

For pre-infection persons, if the sum is greater than a threshold of 400, intervention by a pharmaceutical compound is warranted to lower ACE2 levels. The lowering can be accomplished by administration of one or more of a renin inhibitor and/or a beta blocker, and/or cessation of administration of an ACE inhibitor and/or ARB. As a theoretic example for a pre-infection person with the renin SNP that is positive homozygous for ACE2 levels, the theoretic calculation would be 10×10=100. If 100 would be the threshold for a pre-infection indicating need for a reduction of ACE2 levels (and not the 400 for the full 21 SNP analysis), then a theoretic protocol according to the invention would be to administer a renin inhibitor and a beta blocker. These pharmaceuticals act in the RAAS system to counter or lower ACE2 production. The lowering of the ACE2 level for such a person, will lessen the ability of the coronavirus particles to attack, attach and invade the person's cells through the agency of the ACE2 protein according to aspects of the invention. Similarly, if theoretically the pre-infection person has a renin SNP that is null homozygous, the theoretic calculation would 1×10=10. With 100 as the theoretic threshold, this pre-infection person would not need a reduction in ACE2 levels.

For post-infection patients, if the sum is lower than 550, intervention by a pharmaceutical compound to increase ACE2 levels is warranted. The increase can be accomplished by administration of one or more of ACE inhibitors and/or ARB's and/or beta blockers.

Pharmaceutical Compounds for Augmenting or Reducing ACE2 Levels

The angiotensin converting enzyme inhibitor (ACE inhibitor) useful according to the present invention and pursuant to the foregoing discussion can be selected from enalapril, lisinopril, captopril, alacipril, benazapril, cilazapril, delapril, fosinopril, perindopril, quinapril, ramipril, moveltipril, spirapril, ceronapril, imidapril, temocapril, trandolopril, utilbapril, zofenopril, CV5975, EMD 56855, libenzapril, zalicipril, HOE065, MDL 27088, AB47, DU 1777, MDL 27467A, Equatenr^{M}, Prentyl™, Synecor™, and Y23785 (see WO2010077928A2); and the diuretic is selected from hydrochlorothiazide (HCTZ), furosemide, altizide, trichlormethazide, triflumethazide, bemetizide, cyclothiazide, methylchlothiazide, azosemide, chlorothiazide, butizide, bendroflumethazide, cyclopenthiazide, benzclortriazide, polythiazide, hydroflumethazide, benzthiazide, ethiazide, penflutazide, and any combination thereof.

The angiotensin II receptor antagonists or blockers (ARBs or AT1R/AT2R blockers) useful according to the present invention and pursuant to the foregoing discussion can, for example, be selected from losartan, valsartan, candesartan, irbesartan, olmesartan, azilsartan (Edarbi), eprosartan (Teveten) telmisartan (Micardis), sacubitril, nebivolol, entresto (sacubitril/valsartan), byvalson (nebivolol/valsartan) or any combination thereof.

The renin inhibitors useful according to the present invention and pursuant to the foregoing discussion can be selected from urea derivatives of di- and tri-peptides (See U.S. Pat. No. 5,116,835), amino acids and derivatives (U.S. Pat. Nos. 5,095,119 and 5,104,869), amino acid chains linked by non-peptidic bonds (U.S. Pat. No. 5,114,937), di- and tri-peptide derivatives (U.S. Pat. No. 5,106,835), peptidyl amino diols (U.S. Pat. Nos. 5,063,208 and 4,845,079) and peptidyl beta-aminoacyl aminodiol carbamates (U.S. Pat. No. 5,089,471); also, a variety of other peptide analogs as disclosed in the following U.S. Pat. Nos. 5,071,837; 5,064,965; 5,063,207; 5,036,054; 5,036,053; 5,034,512 and 4,894,437, and small molecule renin inhibitors (including diol sulfonamides and sulfinyls (U.S. Pat. No. 5,098,924). N-morpholino derivatives (U.S. Pat. No. 5,055,466), N-heterocyclic alcohols (U.S. Pat. No. 4,885,292) and pyrolimidazolones (U.S. Pat. No. 5,075,451); also, pepstatin derivatives (U.S. Pat. No. 4,980,283) and fluoro- and chloro-derivatives of statone-containing peptides (U.S. Pat. No. 5,066,643), enalkrein. RO 42-5892. A 65317, CP 80794, ES1005, ES 8891, SQ 34017, aliskiren ((2S,4S,5S,7S)—N-(2-carbamoyl-2-methylpropyl)-5-amino-4-hydroxy-2,7-diisopropyl-8-[4-methoxy-3-(3-methoxypropoxy)phenyl]-oetanamid hemifumarate) SPP600, SPP630 and SPP635), or any combination thereof; the contents of which U.S. Pat. Nos. are specifically incorporated herein by reference in their entireties.

The beta blockers useful according to the present invention and pursuant to the foregoing discussion can be selected from Propranolol, Bucindolol, Carteolol, Carvedilol, Labetalol, Nadolol, Oxprenolol, Penbutolol, Pindolol, Sotalol, Timolol, Acebutolol, Atenolol, Betaxolol, Bisoprolol, Celiprolol, Metoprolol, Metoprolol tartrate, Nebivolol, Esmolol, Butaxamine, ICI-118,551, SR 59230A, Nebivolol and any combination thereof.

Co-administration of antiviral pharmaceuticals along with the foregoing RAAS pharmaceuticals may be warranted for patients exhibiting symptoms of COVID-19 infection. Exemplary antiviral pharmaceuticals include AZT, acyclovir also known as abacavir, lamivudine, raltegravir, elvitegravir, dolutegravir, lopinavir, indinavir, nelfmavir, amprenavir, ritonavir, darunavir or atazanavir or any combination thereof. Co-administration of quinoline and/or naphthoquinone derivatives such as hydroxychloroquine, chloroquine, amodiaquine, mefloquine or atovaquone or any combination thereof may also be warranted to lessen the titer of COVID-19 of an infected patient, the quinoline and naphthoquinone derivatives are commonly known as anti-malarial pharmaceuticals.

Medications that Alter the Renin-Angiotensin-Aldosterone Pathway and Influence ACE2

ACE2 is a relatively recent discovery of a protein/enzyme/receptor factor of the RAAS pathway. Very few pharmacological developments have been made that directly affect the level, activity and expression of ACE2. Facts about ACE2 show how its activity and expression are indirectly alterable.

ACE2 hydrolyzes Ang-I to make Ang (1-9) and Ang-II to make Ang (1-7)[11, 13] and ACE2 has 400-500 fold lower affinity for Ang-1 than for Ang-II. ACE2 does not convert Ang-I to Ang-II, and ACE2 is resistant to ACE-inhibitors. Nevertheless, ACE-inhibition has been shown to increase indirectly ACE2 levels as is expected because of the homeodynamic balance between Ang-II and ACE2 and the ACE conversion of Ang-I to Ang-II. Thus, Ang-II is also a primary target for pharmacotherapy, along with ACE.

Therefore, according to aspects of the invention, positive or negative alterations in Ang-II expression/activity and alterations in ACE expression/activity will positively or negatively alter ACE2 activity. Since ACE2 is also part of the entire RAAS pathway which in turn is dependent on renin, reductions in renin levels, through renin inhibition, or differences in functional variants of renin, will positively or negatively alter ACE2 expression and/or activity. Previous developments by the inventors have demonstrated that the administration of an ACE-inhibitor or an ARB causes a substantial increase in Ang (1-7) levels which indicates augmentation of ACE2 activity.

Health and Genetic Differences Relating to Endogenous ACE2

According to aspects and embodiments of the invention, pharmacologic modulation of Ang-II will impact ACE2 levels because Ang-II on the one hand and ACE2 and Ang (1-7) on the other are in a homodynamic balance. The balance is demonstrated by the relationship of ACE2, Ang-I and Ang-II. ACE2 hydrolyzes both Ang-I and Ang-II but Ang-II is the major substrate for Ang (1-7) synthesis[9]. Evidence shows that ACE2/Ang(1-7) activity functions to counteract the hypertensive effects of Ang-II in patients with high blood pressure[8, 10, 11]. Consequently, developmental aspects according to the invention indicate that elevation of blood pressure leads to an increase in ACE2 expression and/or activity as the body's natural mechanism to counteract the increase and this increase is especially prevalent for hypertensive persons and patients. In addition, ACE2 levels have been shown to be elevated in both type-I and type-II diabetes.

According to aspects and embodiments of the invention, this increase of ACE2 expression and activity leads to an increase in the susceptibility of respiratory pathway cells to the corona-type viruses[16]. The respiratory pathway cells are the lung cells having ACE2 as an integral protein of their cellular membranes. Demonstration of this susceptibility is shown by the most common comorbidities of covid-19: cerebrovascular disease (22 of patients %) and diabetes (22% of patients)[14]; and a second study places these comorbidities at 23.7% and 30% (for hypertension) and 16.2% and 12% (for diabetes)[15].

In addition to hypertension and diabetes being common co-morbidities for the coronaviruses, older individuals and males are more susceptible to the infection (and have demonstrated more severe complications from coronavirus). Both age and sex have previously been shown to be associated with differences in ACE2 levels.

Genetic differences of importance along the renin-angiotensin-aldosterone pathway result in alterations of ACE2 expression and activity and can predict susceptibility to COVID-19 infection. According to the invention, patients who have more severe COVID-19 disease/infection/death rates are in genetic groups associated with higher ACE2 activity. Hence, lowering ACE2 activity in persons who are in danger of becoming infected or have just become infected such as but not limited to one to 2 days will lessen the ability of COVID-19 to invade the respiratory pathway cells that are the target sites for COVID-19. The result will be a lower degree of infection and/or a warding away of infection. Because multiple genes are involved in this RAAS pathway, multiple genes according to the invention are weighted for importance in ACE2 activity and expression. The combination and weighting of the multiple genes involved in ACE2 activity and expression provide the importance and functionality results for reduction of ACE2 expression, function and activity as set forth in Table 1. These are the common genotypes within the RAAS (angiotensinogen, renin, ACE, AT1, and ACE2) that are known to influence protein, enzyme, and receptor function. In addition, the administration of a β-blocker can suppress Ang-II levels and act like either an ACE-inhibitor or A-II receptor blocker on this system.

According to the invention, embodiments and aspects of the invention provide that genotypes of the pi adrenergic receptor (ADRB1) and those along the RAAS pathway should be considered collectively, following individual weighting for their importance in the modulation of ACE2, in order to determine modifications of medications that influence ACE2 levels. Moreover, differential consideration (i.e., decrease or increase of ACE2 activity) is to be made relative to pre and post infection by a coronavirus that utilizes ACE2 as an infection pathway.

The differential consideration for pre-infection involves protocols according to the invention for a lessening or reduction of a person's ACE2 expression, function and/or activity when the person has been exposed to COVID-19 or is in danger of such exposure but has not yet been infected. Table 1 illustrates the correlation among the genotypes, SNPs and pharmaceuticals that will accomplish this reduction of ACE2 activity.

TABLE 1
Target Genotypes to Predict ACE2 Activity and Desired Modification with Pharmacotherapy, Pre-Infection; Pre-
Diagnosis COVID-Type Infection: Reduce ACE2/Ang(1-7) With Genotyping and Alterations of Common Medications

| | | Functional | | | | | Importance | Desired |
|---|---|---|---|---|---|---|---|---|
| Gene | rs# | Allele | Homoz Fx | Heteroz Fx | Null Fx | Function | (1-10) | Genotype |
| Angiotensinogen | | | | | | | | Less |
| (AGT) | | | | | | | | Functional |
| −6 | rs5051 | T (nucleotide) | 10 | 5 | 1 | Increased BP | 6 | |
| | | | | | | AngII infusion, | | |
| | | | | | | greater BP drop | | |
| | | | | | | with ACEi or | | |
| | | | | | | ARB | | |
| M235T | rs699 | C | 10 | 10 | 1 | Higher | 8 | |
| | | | | | | Angiotensinogen | | |
| | | | | | | Levels, BP | | |
| | | | | | | response to ACEi | | |
| | | | | | | and ARB | | |
| T174M | rs4762 | T (encodes | 10 | 5 | 1 | Associated with | 7 | |
| | | Met) | | | | higher BP in | | |
| | | | | | | females, | | |
| | | | | | | increased pre- | | |
| | | | | | | eclampsia, and | | |
| | | | | | | intracerebral | | |
| | | | | | | hemorrhage | | |
| | rs7079 | G | 10 | 1 | 1 | Better BP | 6 | |
| | | | | | | response to ACEi | | |
| Renin | | | | | | | | Less |
| | | | | | | | | Functional |
| | rs12750834 | A | 10 | 10 | 1 | Associated w/ | 10 | |
| | | | | | | plasma renin | | |
| | | | | | | activity (PRA) | | |
| | | | | | | activity, BP | | |
| | | | | | | response to ACEi | | |
| | | | | | | and ARB | | |
| ACE | | | | | | | | More |
| | | | | | | | | Functional |
| I/D | rs1799752 | D | 10 | 10 | 1 | More ACE | 10 | |
| | | | | | | Activity/Ang(1-7) | | |
| | | | | | | 4x Lower in DD - | | |
| | | | | | | Shows Deletion | | |
| | | | | | | decreases ACE2 | | |
| AT1R | | | | | | | | More |
| | | | | | | | | Functional |
| A1166C | rs5186 | C | 10 | 5 | 1 | Hypertension, | 10 | |
| | | | | | | response to ARB | | |
| ACE2 | | | | | | | | Less |
| | | | | | | | | Functional |
| | rs112171234 | G | 10 | 5 | 1 | Alterations in | 8 | |
| | | | | | | ACE2 Activity | | |
| | rs12010448 | C | 10 | 5 | 1 | Alterations in | 8 | |
| | | | | | | ACE2 Activity | | |
| | rs143695310 | A | 10 | 5 | 1 | Alterations in | 8 | |
| | | | | | | ACE2 Activity | | |
| | rs1996225 | T | 10 | 5 | 1 | Alterations in | 8 | |
| | | | | | | ACE2 Activity | | |
| | rs200781818 | Ins | 10 | 5 | 1 | Alterations in | 8 | |
| | | | | | | ACE2 Activity | | |
| | rs2158082 | A | 10 | 5 | 1 | Alterations in | 10 | |
| | | | | | | ACE2 Activity, | | |
| | | | | | | minor allele | | |
| | | | | | | frequency | | |
| | | | | | | difference | | |
| | rs4060 | A | 10 | 5 | 1 | Alterations in | 8 | |
| | | | | | | ACE2 Activity | | |
| | rs4646127 | G | 10 | 5 | 1 | Alterations in | 10 | |
| | | | | | | ACE2 Activity, | | |
| | | | | | | minor allele | | |
| | | | | | | frequency | | |
| | | | | | | difference | | |
| | rs4830983 | C | 10 | 5 | 1 | Alterations in | 8 | |
| | | | | | | ACE2 Activity | | |
| | rs5936011 | T | 10 | 5 | 1 | Alterations in | 10 | |
| | | | | | | ACE2 Activity, | | |
| | | | | | | minor allele | | |
| | | | | | | frequency | | |
| | | | | | | difference | | |
| | rs5936029 | T | 10 | 5 | 1 | Alterations in | 10 | |
| | | | | | | ACE2 Activity, | | |
| | | | | | | minor allele | | |
| | | | | | | frequency | | |
| | | | | | | difference | | |
| | rs6629110 | C | 10 | 5 | 1 | Alterations in | 10 | |
| | | | | | | ACE2 Activity, | | |
| | | | | | | minor allele | | |
| | | | | | | frequency | | |
| | | | | | | difference | | |
| Beta-1 | | | | | | | | Less |
| Adrenergic | | | | | | | | Functional |
| Receptor | | | | | | | | |
| ADRB1 49 | rs1801252 | A | 10 | 5 | 1 | Receptor Density, | 6 | |
| | | | | | | Function, | | |
| | | | | | | Response to β- | | |
| | | | | | | adrenergic | | |
| | | | | | | receptor blockers | | |
| ADRB1_389 | rs1801253 | C | 10 | 5 | 1 | Receptor Density, | 7 | |
| | | | | | | Function, | | |
| | | | | | | Response to β- | | |
| | | | | | | adrenergic | | |
| | | | | | | receptor blockers | | |

Pre-Infection and ACE2 and Aug(1-7) Reduction

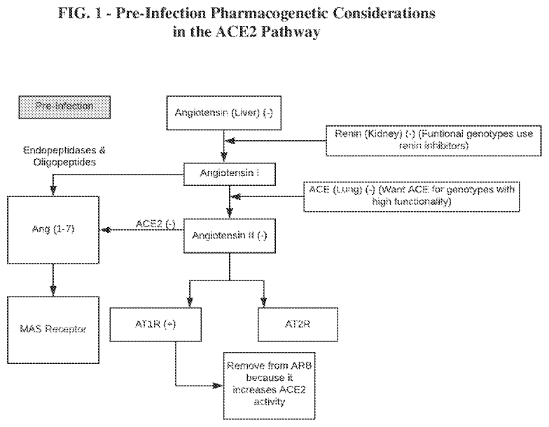

The complex interactions of ACE2 levels along a pathway that is commonly treated with antihypertensive medications show that pharmacotherapy can target certain genetic combinations to attenuate ACE2 levels and reduce the likelihood for severe infections from coronaviruses (FIG. 1). The reverse of attenuation pharmacotherapy is also important to consider. Common antihypertensive medications can lead to an increase in ACE2 levels, particularly in persons not yet infected by COVID-19 and who are genetically prone to high ACE2 levels along the same pathway. These factors are illustrated by FIG. 1.

Post-Infection and ACE2 and Ang(1-7) Augmentation

There is strong evidence that decreased ACE2 and Ang(1-7) levels are associated with poor prognosis in COVID-19 infected patients with respiratory dysfunction, including acute lung injury (ALI) and acute respiratory distress syndrome. The primary cause of hospitalization in COVID-19 cases has been respiratory distress. A large number of distressed COVID-19 patients also demonstrate cardiomyopathy and heart failure. These facts indicate that the protective mechanism of ACE2 and Ang(1-7) in the lungs and the heart is lost, post-infection, and augmentation of this pathway will reduce the severity of the illness, particularly in genetic groups that are susceptible to low ACE2 levels.

The Lung

ACE2 is localized to the airway passage cells of the lungs. e.g., alveoli and similar tissues, as is the angiotensin receptor, type-1 (AT1), but not the angiotensin receptor, type-2. ACE2 and AT1 demonstrate an important interaction for lung protection in models of disease, including those caused by coronaviruses[20-22]. For example, ACE2 knockout mice developed more severe acute lung injury (ALI) with an endotoxin challenge [23]. In models of acute ALI, acid-treated mice who are knockout for ACE2 have more lung elastase, when compared to wild type mice, and this loss of ACE2 resulted in more pronounced deoxygenation, lung edema, increased inflammatory cell infiltration[21]. Additional work has demonstrated that ACE2 decreases fibroblast mitigation in pulmonary fibrosis[24]. Finally, injection of ACE2 seems to protect mice with ALI[21].

Genetic knock-out of AT1a receptor expression markedly improved lung function in Agtr1a−/− mice, confirming the function of Ang-II in lung function. Additional work has shown that pharmacologic inhibition of AT1 receptor attenuates the severity of acid-induced lung injury in ACE2 knockout mice and the loss of ACE2 expression in ALI leads to leaky pulmonary blood vessels through AT1a receptor stimulation[21]. Previous research has demonstrated that the injection of SARS virus in mice leads to an increase in Ang-II levels in lung tissue and exacerbates ALI. Additionally, the viral spike from SARS seems to inhibit ACE2 which increases levels of Ang-II. It is also possible that differences in ACE levels may influence pulmonary function and disease severity via an upstream (of Ang-I) mechanism. In fact, the deletion (D) genotype of ACE is associated with acute respiratory distress syndrome ARDS susceptibility and outcome, where DD genotype is associated with actual acute respiratory distress syndrome ARDS[25]. This same genotype of ACE is also associated with higher ACE activity, which would, theoretically, result in lower Ang-II levels.

The Heart

Previous work in coronaviruses has demonstrated that a high percentage of coronavirus patients with complications have cardiomyopathy. Recent work demonstrated that a greater number of patients hospitalized with COVID-19 demonstrate cardiac injury[26]. This cardiac dysfunction with the viral infection is likely due to a temporary drop in ACE2 levels, as previous evidence indicates a protective role of ACE2 in the heart[27]. For example, the disruption of ACE2 accelerates cardiac hypertrophy and shortens the transition period to heart failure in an Ang-II model of heart failure[28]. In addition, pharmacologic inhibition of ACE2 exacerbates cardiac hypertrophy and circulating Ang(1-7) levels in transgenic rats [29, 30]. The attenuation of Angiotensin-II can help with cardiac remodeling in that both ACE inhibition and the administration of ARBs increase ACE2 activity and/or expression.

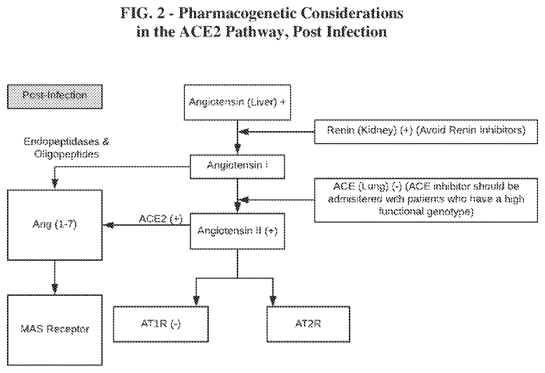

According to aspects of the invention, patients who become infected with COVID-19 lose some of the cardiac and pulmonary protective mechanisms of ACE2 and Ang(1-7), due to viral binding. Patients who have less severe disease/infection/death rates are in genetic groups associated with higher ACE2 activity after infection, particularly when multiple genes are weighted for importance in ACE2 activity and expression and considered simultaneously. This relationship is shown by the algorithm presented by FIG. 2.

According to the invention, embodiments and aspects of the invention provide that genotypes of the pi adrenergic receptor (ADRB1) and those along the RAAS pathway should be considered collectively, following individual weighting for their importance in the modulation of ACE2, in order to determine modifications of medications that influence ACE2 levels. Moreover, differential consideration (i.e., decrease or increase of ACE2 activity) is to be made relative to pre and post infection by a coronavirus that utilizes ACE2 as an infection pathway.

Table 2 provides this post-infection coordination of multiple genes associated with ACE2 and their weighting. Modification, addition, or removal of associated antihypertensive medications will augment ACE2 post-infection and protect the cardiopulmonary system.

TABLE 2
Target Genotypes to Predict ACE2 Activity and Desired Modification with Pharmacotherapy, Post Infection; Post-
Diagnosis Covid-Type Infection: Reduce ACE2/Ang(1-7) with Genotyping and Alterations of Common Medications

| | | Functional | | | | | Importance | Desired |
|---|---|---|---|---|---|---|---|---|
| Gene | rs# | Allele | Homoz Fx | Heteroz Fx | Null Fx | Function | (1-10) | Genotype |
| Angiotensinogen | | | | | | | | More |
| (AGT) | | | | | | | | functional |
| −6 | rs5051 | T | 10 | 5 | 1 | Increased blood | 7 | |
| | | (nucleotide) | | | | pressure (BP) | | |
| | | | | | | AngII infusion, | | |
| | | | | | | greater BP drop | | |
| | | | | | | with ACE | | |
| | | | | | | inhibitors | | |
| | | | | | | (ACEi) or ARB | | |
| M235T | rs699 | C | 10 | 10 | 1 | Higher | 9 | |
| | | | | | | Angiotensinogen | | |
| | | | | | | Levels, BP | | |
| | | | | | | response to | | |
| | | | | | | ACEi and ARB | | |
| T174M | rs4762 | T (encodes | 10 | 5 | 1 | Associated with | 8 | |
| | | Met) | | | | higher BP in | | |
| | | | | | | females, | | |
| | | | | | | increased pre- | | |
| | | | | | | eclampsia, and | | |
| | | | | | | intracerebral | | |
| | | | | | | hemorrhage | | |
| | rs7079 | G | 10 | 1 | 1 | Better BP | 7 | |
| | | | | | | response to | | |
| | | | | | | ACEi | | |
| Renin | | | | | | | | More |
| | | | | | | | | Functional |
| | rs12750834 | A | | 10 | 1 | Associated with | 9 | |
| | | | | | | plasma renin | | |
| | | | | | | activity (PRA) | | |
| | | | | | | activity, BP | | |
| | | | | | | responses to | | |
| | | | | | | ACEi and ARB | | |
| ACE | | | | | | | | Less |
| | | | | | | | | Functional |
| I/D | rs1799752 | D | 10 | 10 | 1 | More ACE | 10 | |
| | | | | | | Activity/Ang(1- | | |
| | | | | | | 7) 4x Lower in | | |
| | | | | | | DD - Shows | | |
| | | | | | | Deletion | | |
| | | | | | | decreases ACE2, | | |
| | | | | | | ACEi increases | | |
| | | | | | | ACE2 | | |
| AT1R | | | | | | | | Less |
| | | | | | | | | Functional |
| A1166C | rs5186 | C | 10 | 5 | 1 | Hypertension | 10 | |
| | | | | | | (HTN), response | | |
| | | | | | | to ARB, Lower | | |
| | | | | | | AT1R - better | | |
| | | | | | | ARDS | | |
| ACE2 | | | | | | | | More |
| | | | | | | | | Functional |
| | rs112171234 | G | 10 | 5 | 1 | Alterations in | 6 | |
| | | | | | | ACE2 Activity | | |
| | rs12010448 | C | 10 | 5 | 1 | Alterations in | 6 | |
| | | | | | | ACE2 Activity | | |
| | rs143695310 | A | 10 | 5 | 1 | Alterations in | 6 | |
| | | | | | | ACE2 Activity | | |
| | rs1996225 | T | 10 | 5 | 1 | Alterations in | 6 | |
| | | | | | | ACE2 Activity | | |
| | rs200781818 | Insertion | 10 | 5 | 1 | Alterations in | 6 | |
| | | (Ins) | | | | ACE2 Activity | | |
| | rs2158082 | A | 10 | 5 | 1 | Alterations in | 8 | |
| | | | | | | ACE2 Activity, | | |
| | | | | | | MAF difference | | |
| | | | | | | Chinese vs | | |
| | | | | | | European | | |
| | rs4060 | A | 10 | 5 | 1 | Alterations in | 6 | |
| | | | | | | ACE2 Activity | | |
| | rs4646127 | G | 10 | 5 | 1 | Alterations in | 8 | |
| | | | | | | ACE2 Activity, | | |
| | | | | | | MAF difference | | |
| | | | | | | Chinese vs | | |
| | | | | | | European | | |
| | rs4830983 | C | 10 | 5 | 1 | Alterations in | 6 | |
| | | | | | | ACE2 Activity | | |
| | rs5936011 | T | 10 | 5 | 1 | Alterations in | 8 | |
| | | | | | | ACE2 Activity, | | |
| | | | | | | MAF difference | | |
| | | | | | | Chinese vs | | |
| | | | | | | European | | |
| | rs5936029 | T | 10 | 5 | 1 | Alterations in | 8 | |
| | | | | | | ACE2 Activity, | | |
| | | | | | | MAF difference | | |
| | | | | | | Chinese vs | | |
| | | | | | | European | | |
| | rs6629110 | C | 10 | 5 | 1 | Alterations in | 8 | |
| | | | | | | ACE2 Activity, | | |
| | | | | | | MAF difference | | |
| | | | | | | Chinese vs | | |
| | | | | | | European | | |
| Beta-1 | | | | | | | | More |
| Adrenergic | | | | | | | | Functional |
| Receptor | | | | | | | | |
| ADRB1_49 | rs1801252 | A | 10 | 5 | 1 | Receptor | 6 | |
| | | | | | | Density, | | |
| | | | | | | Function, | | |
| | | | | | | Response to β- | | |
| | | | | | | blockers | | |
| ADRB1_389 | rs1801253 | C | 10 | 5 | 1 | Receptor | 7 | |
| | | | | | | Density, | | |
| | | | | | | Function, | | |
| | | | | | | Response to β- | | |
| | | | | | | blockers | | |

Polymorphism Detection

According to aspects of the invention, the polymorphisms present in the 21 SNPs of genes which to be determined according to embodiments of the invention, including AGT, Renin, ACE, AT1R, ACE2, ADRB1, can be detected by any available procedure. For example, samples of cDNA, genomic DNA, and/or mRNA can be obtained from a subject and the sequences of polymorphic or variant sites can be evaluated by procedures such as nucleic acid amplification (e.g., PCR), reverse transcription, insertion/deletion analysis, primer extension, probe hybridization, SNP analysis, sequencing, restriction fragment length polymorphism, Matrix-Assisted Laser Desorption/Ionization Time-Of-Flight mass spectrometry (MALDI-TOF MS), Sequenom MassArray genotyping, Sanger sequencing, polyacrylamide gel electrophoresis, agarose gel electrophoresis, probe array hybridization analysis, and combinations thereof.

The methods for detecting polymorphisms can therefore involve detecting an alteration in a nucleic acid. As used herein a “nucleic acid” is a DNA or RNA molecule. A nucleic acid can be a segment of genomic DNA (e.g., an entire gene, an intron of a gene, an exon of a gene, a segment that includes regulatory elements, a 5′ non-coding segment, a 3′ non-coding segment, or any combination thereof). The nucleic acid can also be a cDNA (having exons but not introns), an amplicon, an RNA, a primer, or probe.

Probes and/or primers can be used that can hybridize to nucleic acid segments flanking or including of any of SNPs, insertions, deletions, polymorphic, or other variant segments of AGT, Renin, ACE, AT1R, ACE2, ADRB1. For example, probes and/or primers can be employed that hybridize to nucleic acid segments flanking or including any of the following polymorphisms: rs 5051, rs699, rs4762, rs7079, rs12750834, rs1799752, rs5186, rs112171234, rs12010448, rs143695310, rs1996225, rs200781818, rs2158082, rs4060, rs4646127, rs4830974, rs5936011, rs6629110, rs1801252, rs1801253 which are associated with the above-identified genes as provided on Tables 1 and 2.

For example, the probes and/or primers can separately hybridize to segments of any of these SNPs, as well as to the complementary sequences, amplicons, cDNA, cRNA, and genomic sequences thereof. The probes and/or primers can hybridize to genomic, complementary, amplicon, or cDNA sequences that flank up to 50 nucleotides of any of these SNPs within the corresponding genes on either or both of the 5′ and 3′ sides of the polymorphism.

Methods and devices described herein can detect simultaneously or sequentially all of these polymorphisms. In some embodiments, the methods and devices described herein detect no other polymorphisms, although such methods and devices can include steps and probes for detecting 14 control target nucleic acids. For example, the methods, devices, and kits described herein can detect and evaluate 20 or more polymorphisms.

The probes and primers can be of any convenient length selected by one of skill in the art such as at least 12 nucleotides long, or at least 13 nucleotides long, or at least 14 nucleotides long, or at least 15 nucleotides long, or at least 16 nucleotides long, or at least 17 nucleotides long, or at least 18 nucleotides long, or at least 19 nucleotides long, or at least 20 nucleotides long. In some embodiments, the probes and primers can be less than 150 nucleotides in length, or less than 125 nucleotides in length, or less than 100 nucleotides in length, or less than 75 nucleotides in length, or less than 65 nucleotides in length, or less than 60 nucleotides in length, or less than 55 nucleotides in length, or less than 50 nucleotides in length, or less than 45 nucleotides in length, or less than 40 nucleotides in length.

To detect hybridization, it may be advantageous to employ probes, primers and other nucleic acids in combination with an appropriate detection means. Labels incorporated into primers, incorporated into the amplified product during amplification, or attached to probes that can hybridize to the target, or its amplified product, are useful in the identification of nucleic acid molecules. A number of different labels may be used for this purpose including, but not limited to, fluorophores, chromophores, radiolabels, enzymatic tags, antibodies, chemiluminescence, electroluminescence, and affinity labels. One of skill in the art will recognize that these and other labels can be used with success in this invention.

Examples of affinity labels include but are not limited to the following: an antibody, an antibody fragment, a receptor protein, a hormone, biotin, dinitrophenyl (DNP), or any polypeptide/protein molecule that binds to an affinity label. Examples of enzyme tags include enzymes such as urease, alkaline phosphatase or peroxidase to mention a few. Colorimetric indicator substrates can be employed to provide a detection means visible to the human eye or spectrophotometrically, to identify specific hybridization with complementary nucleic acid-containing samples. Examples of fluorophores include, but are not limited to, Alexa 350, Alexa 430, AMCA, BODIPY 630/650, BODIPY 650/665, BODIPY-FL, BODIPY-R6G, BODIPY-TMR, BODIPY-TRX, Cascade Blue, Cy2, Cy3, Cy5, 6-FAM, Fluorescein, HEX, 6-JOE, Oregon Green 488. Oregon Green 500, Oregon Green 514, Pacific Blue, REG. Rhodamine Green, Rhodamine Red, ROX, TAMRA, TET, Tetramethylrhodamine, and Texas Red.

Means of detecting such labels are well known to those of skill in the art. For example, radiolabels may be detected using photographic film or scintillation counters. In other examples, fluorescent markers may be detected using a photodetector to detect emitted light. In still further examples, enzymatic labels are detected by providing the enzyme with a substrate and detecting the reaction product produced by the action of the enzyme on the substrate, and colorimetric labels are detected by simply visualizing the colored label or by use of spectrometer.

So called “direct labels” are detectable labels that are directly attached to or incorporated into a probe or primer, or to the target (sample) nucleic acid prior to hybridization to a probe that can, for example, be present on a plate, chip, microtiter plate, or microarray. In contrast, so called “indirect labels” are joined to the hybrid duplex after hybridization. In some embodiments, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin-bearing hybrid duplexes providing a label that is easily detected. For a detailed review of methods of labeling nucleic acids and detecting labeled hybridized nucleic acids see, for example, Peter C. van der Vliet & Shiv Pillai, eds., Laboratory Techniques in Biochemistry and Molecular Biology (1993).

Probe arrays, assay plates, and gene chip technology provide a means of rapidly screening a large number of nucleic acid samples for their ability to hybridize to a variety of probes immobilized on a solid substrate that is part of the probe array, assay plate, gene chip or microarray. The technology capitalizes on the complementary binding properties of single stranded nucleic acid probe to screen nucleic acid samples by hybridization (Pease et al., Proc. Natl. Acad. Sci. U.S.A 91: 5022-5026 (1994); U.S. patent to Fodor et al. (1991)). Basically, a nucleic acid probe array or gene chip consists of a solid substrate with an attached array of single-stranded probe molecules. In some embodiments, the probes can fold back on (i.e., hybridize to) themselves to quench a signal from an attached label, but the probes unfold to hybridize to a target nucleic acid, whereupon the signal from the attached label becomes detectable. In other embodiments, the probe can be complementary to the segment of a target nucleic acid but the 3′ end of the probe terminates one nucleotide short of a SNP in the target nucleic acid. The target nucleic acid can be longer than the probe. A signal can be detected upon primer extension of the probe, where the assay mixture contains just one type of labeled nucleotide that can base pair with the variant nucleotide of the SNP. After washing, the presence or absence of the SNP is detectable by incorporation or non-incorporation of the labeled SNP nucleotide into specific probes of the array or plate.

For screening, the chip, plate, or array is contacted with a nucleic acid sample (e.g., genomic DNA, cRNA, cDNA, or amplified copies thereof), which is allowed to hybridize under stringent conditions. The chip, plate, or array is then scanned to determine which targets have hybridized to which probes. The probes are arrayed in known locations so a signal detected at a specific location indicates that its target has hybridized thereto.

Methods for directly synthesizing on or attaching nucleic acid probes to solid substrates are available in the art. See, e.g., U.S. Pat. Nos. 5,837,832 and 5,837,860, both of which are expressly incorporated by reference herein in their entireties. A variety of methods have been utilized to either permanently or removably attach the probes to the substrate. Exemplary methods include: the immobilization of biotinylated nucleic acid molecules to avidin/streptavidin coated supports (Holmstrom, (Anal. Biochem. 209: 278283 (1993)), the direct covalent attachment of short, 5′-phosphorylated primers to chemically modified polystyrene plates (Rasmussen et al., Anal. Biochem. 198: 138-142 (1991)), or the precoating of the polystyrene or glass solid phases with poly-L-Lys or poly L-Lys, Phe, followed by the covalent attachment of either amino- or sulfhydryl-modified oligonucleotides using bifunctional crosslinking reagents (Running et al., BioTechniques 8: 276 277 (1990); Newton, C. R. et al., Acids Res. 21: 1155-1162 (1993)). When immobilized onto a substrate, the probes are typically stabilized and therefore can be used repeatedly.

Hybridization can be performed on an immobilized probe that is attached to a solid surface such as silicon, plastic, nitrocellulose, nylon or glass by addition of one or more target molecules. In some embodiments, the target nucleic acid can be attached to a solid surface and the probe can be added to the immobilized target nucleic acids. Numerous substrate and/or matrix materials can be used, including reinforced nitrocellulose membrane, activated quartz, activated glass, polyvinylidene difluoride (PVDF) membranes, polystyrene, polyacrylamide, poly(vinyl chloride), poly(methyl methacrylate), poly(dimethyl siloxane), photopolymers (which contain photoreactive species such as nitrenes, carbenes and ketyl radicals capable of forming covalent links with target molecules), and combinations thereof.

The term “hybridization” includes a reaction in which one or more polynucleotides react to form a complex that is stabilized via hydrogen bonding between the bases of the nucleotide residues. The hydrogen bonding may occur by Watson-Crick base pairing. Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, primer extension, or the enzymatic cleavage of a polynucleotide by a ribozyme.

Hybridization reactions can be performed under conditions of different “stringency”. The stringency of a hybridization reaction includes the difficulty with which any two nucleic acid molecules will hybridize to one another. Under low to medium stringent conditions, nucleic acid molecules at least 60%, 65%, 70%, 75% identical to each other remain hybridized to each other, whereas molecules with lower percent identity cannot remain hybridized. For detection of single base polymorphisms, higher stringency conditions can be used.

A preferred, non-limiting example of highly stringent hybridization conditions include hybridization in 6×sodium chloride/sodium citrate (SSC) at about 45° C., followed by one or more washes in 0.2×SSC, 0.1% SDS at 50° C., preferably at 55° C., more preferably at 60° C., and even more preferably at 65° C.

When hybridization occurs in an antiparallel configuration between two single-stranded polynucleotides, the reaction is called “annealing” and those polynucleotides are described as “complementary”. A double-stranded polynucleotide can be “complementary” and/or “homologous” to another polynucleotide if hybridization can occur between one of the strands of the first polynucleotide and the second.

Complementarity” or “homology” (the degree that one polynucleotide is identical or complementary to another) is quantifiable in terms of the proportion of bases in opposing strands that are expected to hydrogen bond with each other, according to generally accepted base-pairing rules.

Method for Assaying Patient's SNPs

According to methods and embodiments for the invention, the SNPs listed above may be obtained simultaneously or sequentially through tissue or fluid sampling from the person and/or patient and analysis of the tissue or fluid for the specified SNPs. Typical, known methods for identification of the specified SNPs follows procedures described above for polymorphism detection and is practiced by lysis of the tissue and/or fluid samples, extraction of the DNA, applying PCR amplification techniques using primers to sort and amplify the specified SNPs and analyzing the SNPs by techniques such as mass spectrometric mass array or by chromatographic techniques such as HPLC, electrophoresis, fluorescent labeling and similar techniques.

In particular, practice of the methods for obtaining and identifying the specified SNPs can be accomplished with a PCR thermocycler instrument such as an MJ Research Tetrad 2 thermocycler for PCR amplification and an instrument for identification of the amplified DNA fragments such as an Agena MassArray® system or a Sequenom MALDI-TOF system which utilize the function of a time-of-flight mass spectrometer. A patient's DNA is first extracted as described above and the PCR reaction conducted in the MJ thermocycler to amplify only DNA fragments corresponding substantially to the patient's genomic SNPs described above. The amplified genomic material is then introduced into the detection and identification instrument such as an Agena or Sequenom automated DNA fragment identification system. This system enables identification of multiples of PCR amplified DNA fragments and provides identification of the person or patient's 20 genotype SNPs in accordance with embodiments of the invention.

Example of Sample Processing

Each patient is given a collection kit consisting of two buccal swabs and two uniquely barcoded tubes (termed A and B swabs) containing a proprietary lysis buffer consisting of 50 mM Tris pH 8.0. 50 mM EDTA, 25 mM Sucrose, 100 mM NaCl, and 1% SDS. The patient will use the swab to collect buccal cells by scraping the inside of their cheek and place the swab in the provided barcoded tube, one swab for each cheek. Once the swab has been placed into the lysis buffer the cells are no longer viable and therefore samples are now considered to be nucleic acids and safe to be shipped via standard mail. All samples are checked-in. The barcodes of the samples are scanned and their arrival in the laboratory is confirmed.

In general, two samples (Swab A and Swab B) are taken. The Swab A sample is subjected to the process (DNA Extraction through Reporting) unless the Swab A sample fails either the DNA Yield and Purity Assays. Genomic Analysis, or the PCR QA Assay. If such failure occurs, then the other sample (Swab B) is subjected to the process.

The samples are grouped into sets of 91 and assigned positions in 96 sample grids (12×8 grid layout) for DNA extraction. The remaining five positions in each grid can be extraction controls (four negative controls [H₂O] and one non-human positive). The five controls can be assigned random positions in each grid, giving each grid/plate a unique “plate fingerprint.” The randomly assigned controls prevent possible plate swaps or 180° rotations as every plate is now identifiable simply by control positions. All samples are then normalized to a volume of 650 ul by addition of the above mentioned lysis buffer. Additionally, 25 ul of proteinase K (ProK) is added and each sample is incubated in a 55° C. oven for a minimum of 4 hours.

Following such incubation, the samples are extracted using a BioSprint96 (KingFisher96) Robotic workstation with magnetic-particle DNA purification chemistry to isolate genomic DNA (GenomicDNA) from tissue samples. This protocol utilizes the chemistry from the eVoMagDNA Extraction KF96 Kit (Verde Labs, Marietta, GA) and is run to specifications provided by the manufacturer.

Following DNA extraction and subsequent desiccation, the DNA is resuspended in HPLC water. Five microliters of each sample is then aliquoted to assay plates for the first pair of QA assays, both a PicoGreen fluorometric quantification and a spectrophotometric purity estimation. The fluorescence and absorbance data is analyzed for all samples in the 96 well plate, including the five controls. The positions of the negative controls is confirmed and accessed for possible plate contamination. The results for the positive control as well as the samples on the plate are analyzed for quality metrics using a systems analysis approach. The outliers are statistically assessed. After the quantification and purity evaluations. QA assay robotic systems are used to transfer the samples into racks of 96 sample septa sealed plates (to ensure there is no evaporative loss) and a fractional volume of each sample is used to create a daughter plate of the samples at a normalized concentration of 5 ng/μl for the PCR QA assays and subsequent genotyping. The creation of the normalized daughter plate serves two purposes. First, it allows the immediate storage of the primary stock of each sample at −80° C. avoiding the need for unnecessary freeze-thaw of samples and the potential contamination risks associated with repeated accessing of the stock. Second, it avoids unnecessary waste of the DNA associated with the use of full concentration stock for the PCR applications (this −80° C. stock DNA can be used at any time or saved for future testing).

Any samples that fail any of the QA assays can re-enter the pipeline and be sorted and re-processed from the B-swab, which is the second tube/swab in the kit sent to the customer mentioned above. By always having a backup sample it is not necessary to go back to the customer to ask for a re-swab. If the quantity and purity are still insufficient then whole genome amplification and/or organic re-extraction can be employed.

Following the passage of the QA thresholds normalized fractions of the samples are transferred to PCR plates for genotyping. Each sample is analyzed using three different methodologies, the Sequenom MassArray genotyping platform, Sanger sequencing using the ABI 3730xl genomic analyzer from Applied Biosystems, and classical PCR and gel sizing to determine insertion/deletion status. The Sequenom MassArray genotyping platform is used to analyze the following SNP sites: rs 5051, rs699, rs4762, rs7079, rs12750834, rs1799752, rs5186, rs112171234, rs12010448, rs143695310, rs1996225, rs200781818, rs2158082, rs4060, rs4646127, rs4830974, rs5936011, rs6629110, rs1801252, rs1801253.

### REFERENCES

1. Wan, Y., et al., Receptor Recognition by the Novel Coronavirus from Wuhan: an Analysis Based on Decade-Long Structural Studies of SARS Coronavirus. J Virol, 2020. 94(7).
2. Perlman, S. and J. Netland, Coronaviruses post-SARS: update on replication and pathogenesis. Nat Rev Microbiol, 2009. 7(6): p. 439-50.
3. Li. F., Structure, Function, and Evolution of Coronavirus Spike Proteins.

Annu Rev Virol, 2016. 3(1): p. 237-261.
4. Li, W., et al., Angiotensin-converting enzyme 2 is a functional receptor for the SARS coronavirus. Nature, 2003. 426(6965): p. 450-4.
5. Li, W., et al., Efficient replication of severe acute respiratory syndrome coronavirus in mouse cells is limited by murine angiotensin-converting enzyme 2. J Virol, 2004. 78(20): p. 11429-33.
6. Li, W., et al., Receptor and viral determinants of SARS-coronavirus adaptation to human ACE2. EMBO J. 2005. 24(8): p. 1634-43.
7. Bernardi, S., et al., Angiotensin-converting enzyme 2 regulates renal atrial natriuretic peptide through angiotensin-(1-7). Clin Sci (Lond), 2012. 123(1): p. 29-37.
8. Tikellis, C. and M. C. Thomas, Angiotensin-Converting Enzyme 2 (ACE2) Is a Key Modulator of the Renin Angiotensin System in Health and Disease. Int J Pept, 2012. 2012: p. 256294.
9. Chappell, M. C., Emerging evidencefor afunctional angiotensin-converting enzyme 2-angiotensin-(1-7)-MAS receptor axis: more than regulation of blood pressure? Hypertension, 2007. 50(4): p. 596-9.
10. Ferreira, A. J., et al., New cardiovascular and pulmonary therapeutic strategies based on the Angiotensin-converting enzyme 2/angiotensin-(1-7)/mas receptor axis.

Int J Hypertens, 2012. 2012: p. 147825.
11. Ferreira, A. J., R. A. Santos, and M. K. Raizada, Angiotensin-(1-7)/angiotensin-converting enzyme 2/mas receptor axis and related mechanisms. Int J Hypertens, 2012. 2012: p. 690785.
12. Baginski, L., et al., Reverse transcription polymerase chain reaction (RT-PCR) analysis of proteolytic enymes in cultures of human respiratory epithelial cells. J Aerosol Med Pulm Drug Deliv, 2011. 24(2): p. 89-101.
13. Hamming, I., et al., Tissue distribution of ACE2 protein, the functional receptor for SARS coronavirus. A first step in understanding SARS pathogenesis. J Pathol, 2004.203(2): p. 631-7.
14. Yang, X., et al., Clinical course and outcomes of critically ill patients with SARS-CoV-2 pneumonia in Wuhan, China: a single-centered, retrospective, observational study. Lancet Respir Med, 2020.
15. Zhang, J. J., et al., Clinical characteristics of 140 patients infected with SARS-(CoV-2 in Wuhan, China. Allergy, 2020.
16. Bai, F., et al., Angiotensin II AT1 receptor alters ACE2 activity, eNOS expression and CD44-hyaluronan interaction in rats with hypertension and myocardial fbrosis. Life Sci. 2016. 153: p. 141-52.
17. Wysocki, J., et al., ACE and ACE2 activity in diabetic mice. Diabetes, 2006. 55(7): p. 2132-9.
18. Chen, K., et al., Sex-Specific Changes in Renal Angiotensin-Converting Enzyme and Angiotensin-Converting Enzyme 2 Gene Expression and Enzyme Activity at Birth and Over the First Year of Life. Reprod Sci, 2016. 23(2): p. 200-10.
19. Liu. J., et al., Sex differences in renal angiotensin converting enzyme 2 (ACE2) activity are 17beta-oestradiol-dependent and sex chromosome-independent. Biol Sex Differ, 2010. 1(1): p. 6.
20. Kuba, K., et al., A crucial role of angiotensin converting enzyme 2 (ACE2) in SARS coronavirus-induced lung injury. Nat Med, 2005. 11(8): p. 875-9.
21. Imai. Y., et al., Angiotensin-converting enzyme 2 protects from severe acute lung failure. Nature, 2005. 436(7047): p. 112-6.
22. Wu, Y., Compensation of ACE2 Function for Possible Clinical Management of 2019-nCoV-Induced Acute Lung Injury. Virol Sin, 2020.
23. Nagase. T., et al., Acute lung injury by sepsis and acid aspiration: a key role for cytosolic phospholipase A2. Nat Immunol. 2000. 1(1): p. 42-6.
24. Meng, Y., et al., The angiotensin-converting enzyme 2/angiotensin (1-7)/Mas axis protects against lung fibroblast migration and lung fibrosis by inhibiting the NOX4-derived ROS-mediated RhoA/Rho kinase pathway. Antioxid Redox Signal, 2015. 22(3): p. 241-58.
25. Marshall, R. P., et al., Angiotensin converting enzyme insertion/deletion polymorphism is associated with susceptibility and outcome in acute respiratory distress syndrome. Am J Respir Crit Care Med, 2002. 166(5): p. 646-50.
26. Shi, S., et al., Association of Cardiac Injury With Mortality in Hospitalized Patients With COVID-19 in Wuhan. China. JAMA Cardiol, 2020.
27. Ferreira, A. J., et al., Angiotensin-converting enzyme 2 activation protects against hypertension-induced cardiac fibrosis involving extracellular signal—regulated kinases. Exp Physiol, 2011. %(3): p. 287-94.
28. Yamamoto. K., et al., Deletion of angiotensin-converting enzyme 2 accelerates pressure overload-induced cardiac dysfunction by increasing local angiotensin II. Hypertension, 2006. 47(4): p. 718-26.
29. Santos, R. A., et al., Expression of an angiotensin-(1-7)-producing fusion protein produces cardioprotective effects in rats. Physiol Genomics, 2004. 17(3): p. 292-9.
30. Nadu, A. P., et al., Reduced isoproterenol-induced renin-angiotensin changes and extracellular matrix deposition in hearts of TGR(AJ-7)3292 rats. J Am Soc Hypertens, 2008. 2(5): p. 341-8.

### SUMMARY STATEMENTS

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods, devices, and kits described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a polypeptide” includes a plurality of such nucleic acids or polypeptides (for example, a solution of nucleic acids or polypeptides or a series of nucleic acid or polypeptide preparations), and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

1. A method comprising treating a person who is exposed to a coronavirus but is not yet infected with the coronavirus, comprising administering one or more pharmaceutical compounds that lowers ACE2 levels according to one of Pre-infection protocol 1-6 and the person's genotype for a series of 21 single nucleotide polymorphisms (SNPs) recited below as rs numbers with variable nucleotides indicated in parenthesis:
Pre-infection protocol 1 comprises administering a renin inhibitor and/or beta blocker, and discontinuing administration of an ACE inhibitor and/or an angiotensin receptor blocker (ARB) for the person with genotype:
a) angiotensinogen (AGT) SNPs rs5051(T), rs699(C) positive homozygous alleles or heterozygous alleles, and rs4762(T) and rs7079(G) positive homozygous alleles;
b) renin (REN) SNP rs12750834(A) positive homozygous alleles or heterozygous allele;
c) angiotensin-converting enzyme (ACE) SNP rs1799752(Deletion) positive homozygous alleles or heterozygous alleles;
d) angiotensin converting enzyme 2 (ACE2) modulating SNPs include three or more positive homozygous alleles selected from the group rs112171234(G), rs12010448(C), rs143695310(A), rs1996225(T), rs200781818(ins), rs2158082(A), rs4060(A), rs4646127(G), rs4830983(C), rs5936011(T), rs5936029(T), and rs6629110(C);
e) angiotensin receptor type-1 (AT1R) SNP rs5186(C) positive homozygous alleles; and
f) beta-i adrenergic receptor (ADRB1) SNP rs1801252(A) or SNP rs1801253(C) homozygous or heterozygous;
Pre-infection protocol 2 comprises administering a renin inhibitor and/or beta blocker, and discontinuing administration of an ARB for the person with genotype:
a) REN SNP rs12750834(A) positive homozygous alleles or heterozygous alleles,
b) AT1R SNP rs5186(C) positive homozygous alleles, and
c) ADRB1 SNP rs1801252(A) and rs1801253(C) positive homozygous alleles or heterozygous alleles; and
d) AGT, ACE, ACE2, and ADRB1 SNPs null homozygous alleles;
Pre-infection protocol 3 comprises administering a renin inhibitor and/or a beta blocker and/or an ACE inhibitor, and discontinuing administration of an ARB for the person with genotype:
a) AGT SNPs rs5051(T), rs699(C) positive homozygous alleles or heterozygous alleles, rs4762(T) and rs7079(G);
b) REN SNP rs12750834(A) positive homozygous alleles or heterozygous alleles;
c) AT1R SNP rs5186(C) positive homozygous alleles; and
d) ACE SNPs null homozygous alleles;
Pre-infection protocol 4 comprises administering no pharmaceutical compound and discontinuing administration of an ACE inhibitor and/or an ARB for the person with genotype: ACE SNP rs1799752(Deletion) positive homozygous alleles or heterozygous alleles;
Pre-infection protocol 5 comprises administering a renin inhibitor, and discontinuing administration of one or more ACE inhibitors and/or ARBs and/or beta blockers for the person with genotype score greater than 400, wherein the genotype score is determined by obtaining the multiplication product of the weight number of each of the person's individual SNPs weight numbers determined by their positive homozygous fx, heterozygous fx and null fx numbers of Table 1 times the corresponding individual SNP influence numbers of Table 1 and the products are summed together to provide the genotype score;
Pre-infection protocol 6 comprises administering no pharmaceutical compound that lowers ACE2 levels for the person with a genotype different from the genotypes reciting for Pre-infection protocols 1-5.

2. A method according to claim 1 wherein the coronavirus is Covid-19 virus.

3. A method according to claim 1 wherein the blood pressure and blood sugar of the person is evaluated prior to or in conjunction with determining the person's or genotype.

4. A method according to claim 1, wherein blood pressure and blood sugar are evaluated prior to determining the person or patient's genotype and if either is above normal values, the genotype is determined.

5. A method according to claim 1, wherein the age of the person or patient is 60 years or older.

6. A method according to claim 1, wherein the person or patient is a male.

7. The method according to claim 1, wherein the renin inhibitor is selected from urea derivatives of di-peptides and tri-peptides, amino acids and, amino acid chains linked by non-peptidic bonds, di- and tri-peptide derivatives, peptidyl amino diols, peptidyl beta-aminoacyl aminodiol carbamates, a peptide analog, a small molecule renin inhibitors, an N-morpholino derivative, an N-heterocyclic alcohol, a pyrolimidazolones, a pepstatin derivative, a fluoro-derivative of a statone-containing peptide, a chloro-derivative of a statone-containing peptide, enalkrein, RO 42-5892, A 65317, CP 80794, ES1005, ES 8891, SQ 34017, aliskiren ((2S,4S,5S,7S)—N-(2-carbamoyl-2-methylpropyl)-5-amino-4-hydroxy-2,7-diisopropyl-8-[4-methoxy-3-(3-methoxypropoxy)phenyl]-oetanamid hemifumarate) SPP600, SPP630 and SPP635, or any combination thereof.

8. The method according to claim 1, wherein the beta blocker is selected from Propranolol, Bucindolol, Carteolol, Carvedilol, Labetalol, Nadolol, Oxprenolol, Penbutolol, Pindolol, Sotalol, Timolol, Acebutolol, Atenolol, Betaxolol, Bisoprolol, Celiprolol, Metoprolol, Metoprolol tartrate, Nebivolol, Esmolol, Butaxamine, ICI-118,551, SR 59230A, Nebivolol and any combination thereof.

9. The method according to claim 1, wherein the ACE inhibitor is selected from enalapril, lisinopril, captopril alacipril, benazapril, cilazapril, delapril, fosinopril, perindopril, quinapril, ramipril, moveltipril, spirapril, ceronapril, imidapril, temocapril, trandolopril, utilbapril, zofenopril, CV5975, EMD 56855, libenzapril, zalicipril, HOE065, MDL 27088, AB47, DU 1777, MDL 27467A, Equaten™, Prentyl™, Synecor™, and Y23785; and the diuretic is selected from hydrochlorothiazide (HCTZ), furosemide, altizide, trichlormethazide, triflumethazide, bemetizide, cyclothiazide, methylchlothiazide, azosemide, chlorothiazide, butizide, bendroflumethazide, cyclopenthiazide, benzclortriazide, polythiazide, hydroflumethazide, benzthiazide, ethiazide, penflutazide, and any combination thereof.

10. The method according to claim 1, wherein the ARB is selected from losartan, valsartan, candesartan, irbesartan, olmesartan, azilsartan (Edarbi), eprosartan (Teveten) telmisartan (Micardis), sacubitril, nebivolol, entresto (sacubitril/valsartan), byvalson (nebivolol/valsartan) or any combination thereof.

11. A method according to claim 1, wherein a mineralocorticoid receptor antagonist administration is discontinued for Pre-infection protocols 1-5 if the person has been receiving a mineralocorticoid receptor antagonist.

12. A method for treating a patient who is infected with a coronavirus, comprising administering one or more pharmaceutical compounds that increase ACE2 levels according to one of Post-infection protocols 1-6 and the person's genotype for a series of 21 single nucleotide polymorphisms (SNPs) recited below as rs numbers with variable nucleotides indicated in parenthesis:
Post-infection protocol 1 comprises administering an ACE inhibitor and/or ARB for the person with genotype:
a. AGT SNPs rs5051(A) null functional homozygous alleles or heterozygous alleles, rs699(G) null functional homozygous alleles, rs4762(A) null functional homozygous alleles or heterozygous alleles, and rs7079(C) null functional homozygous alleles or heterozygous alleles;
b. REN SNP rs12750834(T) null functional homozygous alleles;
c. AT1R SNP rs5186(T) null functional homozygous alleles or heterozygous alleles;
d. ACE2 SNPs including any three or more null functional homozygous alleles selected from rs112171234(C), rs12010448(G), rs143695310(T), rs1996225(A), rs200781818(Deletion), rs2158082(T), rs4060(T), rs4646127(C), rs4830983(G), rs5936011(A), rs5936029(A), and rs6629110(G); and
e. ADRB1 SNP rs1801252(T) null functional alleles or rs1801253(G) as null functional alleles; and
f. ACE SNP rs1799752(Deletion) positive homozygous alleles or heterozygous alleles;
Post-infection protocol 2 comprises administering an ARB and/or a beta blocker for the person with genotype:
a. REN SNP rs12750834(A) positive homozygous alleles or heterozygous alleles;
b. ADRB1 SNPs rs1801252(A) and rs1801253(C) positive homozygous alleles;
c. AT1R SNP rs5186(C) positive homozygous alleles; and
d. AGT, ACE2, and ACE SNPs heterozygous alleles;
Post-infection protocol 3 comprises administering an ARB for the person with genotype:
a. AGT SNP rs5051(T) positive homozygous alleles, rs699(C) positive homozygous alleles or heterozygous alleles, rs4762(T) positive homozygous alleles, and rs7079(G) positive homozygous alleles;
b. REN SNP rs12750834(A) positive homozygous alleles or heterozygous alleles;
c. AT1R SNP rs5186(A) positive homozygous alleles;
d. ADRB1 SNP either as (rs1801252(T) null homozygous alleles or as rs1801253(G) null homozygous alleles, and;
e. ACE and ACE2 SNPs as heterozygous alleles;
Post-infection protocol 4 comprises administering an ACE inhibitor and/or an ARB for the person with genotype:
a. ACE SNP rs1799752(Deletion) positive homozygous alleles or heterozygous alleles;
b. AT1a SNP rs5186(A) positive homozygous alleles;
c. ACE2 SNPs including three or more null homozygous alleles selected from rs112171234(C), rs12010448(G), rs143695310(T), rs1996225(A), rs200781818(Deletion), rs2158082(T), rs4060(T), rs4646127(C), rs4830983(G), rs5936011(A), rs5936029(A) and rs6629110(G); and
d. AGT, REN, or ADRB1 SNPs as heterozygous alleles;
Post-infection protocol 5 comprises administering one or more of ACE inhibitors and/or ARBs and/or beta blockers for the person with genotype score less than 550, wherein the genotype score is determined by obtaining the multiplication product of the weight number of each of the person's individual SNPs weight numbers determined by their positive homozygous fx, heterozygous fx and null fx numbers of Table 2 times the corresponding individual SNP influence numbers of Table 2 and the products are summed together to provide the genotype score;
Post-infection protocol 6 comprises administering no pharmaceutical compound to the person with a genotype different from the genotypes recited for Post-infection protocols 1-5.

13. A method according to claim 12, wherein the coronavirus is Covid-19.

14. A method according to claim 12, wherein the blood pressure and blood sugar of the patient is evaluated prior to or in conjunction with determining the patient's genotype.

15. The method according to claim 12, wherein blood pressure and blood sugar are evaluated prior to determining the person or patient's genotype and if either is above normal values, the genotype is determined.

16. The method according to claim 12, wherein the age of the person or patient is 60 years or older.

17. The method according to claim 12, wherein the person or patient is a male.

18. The method according to claim 12, wherein the person or patient is asymptomatic but assays positive for Covid-19 infection.

19. The method according to claim 12, further comprising administering an antiviral pharmaceutical, quinoline-like pharmaceutical, naphthoquinone-like pharmaceutical, or a combination thereof.

20. The method according to claim 19, wherein the antiviral pharmaceutical is AZT, acyclovir also known as abacavir, lamivudine, raltegravir, elvitegravir, dolutegravir, lopinavir, indinavir, nelfinavir, amprenavir, ritonavir, darunavir or atazanavir or any combination thereof.

21. The method according to claim 19, wherein the quinoline-like or naphthoquinone-like pharmaceutical is hydroxychloroquine, chloroquine, amodiaquine, mefloquine or atovaquone, or any combination thereof.

22. The method according to claim 19, wherein the antiviral, quinoline-like and/or naphthoquinone-like pharmaceuticals are co-administered.

23. The method according to claim 12, wherein the renin inhibitor is selected from urea derivatives of di-peptides and tri-peptides, amino acids and, amino acid chains linked by non-peptidic bonds, di- and tri-peptide derivatives, peptidyl amino diols, peptidyl beta-aminoacyl aminodiol carbamates, a peptide analog, a small molecule renin inhibitors, an N-morpholino derivative, an N-heterocyclic alcohol, a pyrolimidazolones, a pepstatin derivative, a fluoro-derivative of a statone-containing peptide, a chloro-derivative of a statone-containing peptide, enalkrein, RO 42-5892, A 65317, CP 80794, ES1005, ES 8891, SQ 34017, aliskiren ((2S,4S,5S,7S)—N-(2-carbamoyl-2-methylpropyl)-5-amino-4-hydroxy-2,7-diisopropyl-8-[4-methoxy-3-(3-methoxypropoxy)phenyl]-oetanamid hemifumarate) SPP600, SPP630 and SPP635, or any combination thereof.

24. The method according to claim 12, wherein the beta blocker is selected from Propranolol, Bucindolol, Carteolol, Carvedilol, Labetalol, Nadolol, Oxprenolol, Penbutolol, Pindolol, Sotalol, Timolol, Acebutolol, Atenolol, Betaxolol, Bisoprolol, Celiprolol, Metoprolol, Metoprolol tartrate, Nebivolol, Esmolol, Butaxamine, ICI-118,551, SR 59230A, Nebivolol and any combination thereof.

25. The method according to claim 12, wherein the ACE inhibitor is selected from enalapril, lisinopril, captopril alacipril, benazapril, cilazapril, delapril, fosinopril, perindopril, quinapril, ramipril, moveltipril, spirapril, ceronapril, imidapril, temocapril, trandolopril, utilbapril, zofenopril, CV5975, EMD 56855, libenzapril, zalicipril, HOE065, MDL 27088, AB47, DU 1777, MDL 27467A, Equaten™, Prentyl™, Synecor™, and Y23785; and the diuretic is selected from hydrochlorothiazide (HCTZ), furosemide, altizide, trichlormethazide, triflumethazide, bemetizide, cyclothiazide, methylchlothiazide, azosemide, chlorothiazide, butizide, bendroflumethazide, cyclopenthiazide, benzclortriazide, polythiazide, hydroflumethazide, benzthiazide, ethiazide, penflutazide, and any combination thereof.

26. The method according to claim 12, wherein the ARB is selected from losartan, valsartan, candesartan, irbesartan, olmesartan, azilsartan (Edarbi), eprosartan (Teveten) telmisartan (Micardis), sacubitril, nebivolol, entresto (sacubitriVvalsartan), byvalson (nebivolol/valsartan) or any combination thereof.

27. A method according to claim 12, wherein a mineralocorticoid receptor antagonist is co-administered for Post-Infection Protocols 1-5.
